(19) United States
(12) Patent Application Publication
(10) Pub. No.: US 2023/0138103 A1
(43) Pub. Date: May 4, 2023

(54) GENE SILENCING AGENTS FOR TARGETING CORONAVIRUS GENES AND USES THEREOF

(71) Applicant: 1GLOBE HEALTH INSTITUTE LLC, Norwood, MA (US)

(72) Inventors: Chiang J. LI, Cambridge, MA (US); Xiangao SUN, Brookline, MA (US); Charles LI, Cambridge, MA (US); Pingping "Peter" KUANG, Lexington, MA (US)

(21) Appl. No.: 17/912,510

(22) PCT Filed: Mar. 18, 2021
(86) PCT No.: PCT/US21/23043

§ 371 Date: Sep. 18, 2022

Related U.S. Application Data
(60) Provisional application No. 62/991,580, filed on Mar. 18, 2020.
(60) Provisional application No. 63/092,801, filed on Oct. 16, 2020.

(51) Int. Cl. C12N 15/113

(52) U.S. Cl. CPC C12N 15/1131; C12N 2310/14

(57) ABSTRACT

The present invention provides gene silencing agents that are capable of silencing the expression of the Coronavirus gene(s), e.g., SARS-CoV-2, both in vitro and in vivo. The present invention further provides general and specific compositions and methods of using such compositions that can be used to reduce specific SARS-CoV-2 protein levels or SARS-CoV-2 titers, in a subject, e.g., a mammal such as a human, for research or therapeutic purposes, for example to manage or treat conditions related to Coronavirus infection, e.g., COVID-19.

### CROSS-REFERENCE TO RELATED APPLICATIONS

This application claims priority to and the benefit of co-pending U.S. provisional patent application Serial Nos. 62/991,580, filed Mar. 18, 2020, and 63/092,801, filed Oct. 16, 2020, which applications are incorporated herein by reference in their entirety.

### TECHNICAL FIELD

The invention generally relates to the field of gene silencing agents for use in targeting Coronavirus (CoV) gene(s), e.g., SARS-CoV-2, and methods of using such gene silencing agents to reduce Coronavirus protein levels or viral titers both in vitro and in vivo, and methods of treating subjects having a Coronavirus infection, e.g., COVID-19, or methods of preventing Coronavirus infection.

### BACKGROUND OF THE INVENTION

The COVID-19 that has been gripping the world in recent times belongs to a myriad of single-stranded RNA that caused the Middle East Respiratory syndrome (MERS) in 2012 and severe acute respiratory syndrome (SARS) in 2003, both of which were transmitted to humans from animal origin, emergency medical solution is needed for this severe respiratory infection (Zhu, N., 2019).

COVID-19 is mild for 81% of patients and has an overall case fatality rate of 2-3%. The severity of the disease appears to be associated with age, with the elderly mostly at risk; those over 80 years of age have a Case Fatality Rate (CFR) of 15%. The CFR is also increased in those with comorbidities including cardiovascular, diabetes, chronic respiratory disease, hypertension, and cancer. The cause of death is respiratory failure, shock or multiple organ failure host (Fisher and Heymann, 2020).

No specific therapies have been proved can inhibit CoVs replication in the majority of patients at the present. Gene silencing technology is considered as one of the most promising drug development technology, including ASO (single-stranded antisense oligonucleotide) and RNAi (double-stranded interfering RNA). Rational designed aiRNA, siRNA, ASO or other gene silencing agents targeting the genes could be useful in the treatment of CoV infections.

Asymmetrical interfering RNA (aiRNA) is a class of double-stranded RNA molecules and operating within the RNA interference (RNAi) pathway. It interferes with the expression of specific genes with complementary nucleotide sequences by degrading mRNA after transcription, preventing translation (Sun, X, et al 2008). Emphasized here is that RNAi could be a promising approach in managing the current outbreak of COVID-19. At present, there is no specific treatment option available for advanced COVID-19. While emerging vaccines should limit the propagation of SARS-CoV-2 virus and eventually control the communal spread of the COVID-19disease, there is urgent unmet medical need for therapeutics for those who have already developed the COVID-19 diseases to prevent mortality and alleviate morbidity. Designing virus-targeted small molecules have the drawback that it takes several years to optimize the lead molecule that has an acceptable level of biological function with the ability to reach the target site (Kunisaki, K. M., 2009, and Hodgson, 1, 2020). Also, to develop effective small-molecule therapeutics, such as protease inhibitors/nucleoside analogs, and vaccines, researchers need to know the structural and functional aspects of the virus (Lu, H., 2020). The constraints posed by the above options can be overcome by using aiRNAs, which can target non-druggable targets (Levanova, A., 2018 and Lam, 1K, 2015) with an additional advantage in terms of our ability to rapidly and precisely design aiRNA-based therapeutics using advanced bioinformatic tools. Therefore, there is an urgent need for an effective gene silencing agent such as aiRNAs against the CoV infection, especially against SARS-CoV-2 infection.

### SUMMARY

The present invention provides gene silencing agents that have been shown to silencing CoV genes, in vitro and in vivo.

In one aspect, the present invention specifically provides gene silencing agents comprising an antisense strand consisting of, consisting essentially of or comprising at least 15 or more contiguous nucleotides substantially complementary to one of the genes of CoV, particularly the PL1, 3CL, RdRp (also be referred to as POL or P hereinafter) and Nucleocapsid (N) genes of SARS-CoV-2, and more particularly agents comprising 15 or more contiguous nucleotides substantially complementary to one of the target sequences provided in FIGS. 1-3 (SEQ ID NOS. 1-210). The gene silencing agents preferably comprise an antisense strand and a sense strand, having less than 30 nucleotides per strand, e.g., 9-23 nucleotides.

In another aspect, the present invention specifically provides gene silencing agents comprise an antisense strand consisting of, consisting essentially of or comprising at least 15 or more contiguous nucleotides differing by no more than 3 nucleotides from one of the antisense strand sequences provided in FIGS. 1-3 (SEQ ID NOS. 211-420).

In some embodiments, the gene silencing agents preferably is a double-stranded RNAi agent comprising an antisense strand and a sense strand, having less than 30 nucleotides per strand, e.g., 9-23 nucleotides, such as those provided in FIGS. 1-3 (GHI_N1 to GHI_N96, GHI_P97 to GHI_P162, aiRNA 3CL_1 to 28 and aiRNA PLPRO_1 to 20). The double-stranded RNAi agent can either have blunt end(s) or have overhangs of 1-5 nucleotides at the 3′ and/or 5′ end(s) of the antisense strand of the agent.

In one aspect, the present invention provides double-stranded gene silencing agents comprising an antisense strand and a sense strand forming a double-stranded region, wherein said antisense strand comprises 5′-AACGAUUGCAGCAUUGUUAGC-3′ (SEQ ID NO:253), and said sense strand comprises 5′-AACAAUGCUGCAAUC-3′ (SEQ ID NO:463). The present invention also provides gene silencing agents comprising sense and antisense nucleotide sequences which are at least 90%, 91%, 92%, 93%, 94%, 95%, 96%, 97%, 98%, or 99% identical over their entire length to the foregoing sense and antisense nucleotide sequences.

In one aspect, the present invention provides gene silencing agents, wherein the gene silencing agent is a double-stranded RNAi agent comprising an antisense strand and a sense strand forming a double-stranded region, wherein said antisense strand comprises 5′-AACUUUAUCAUUGUAGAUGUC-3′ (SEQ ID NO:365), and said sense strand comprises 5′-AUCUACAAUGAUAAA-3′ (SEQ ID NO:575). The present invention also provides gene silencing agents comprising sense and antisense nucleotide sequences which are at least 90%, 91%, 92%, 93%, 94%, 95%, 96%, 97%, 98%, or 99% identical over their entire length to the foregoing sense and antisense nucleotide sequences.

In one aspect, the present invention provides gene silencing agents, wherein the gene silencing agent is double-stranded RNAi agent comprising an antisense strand and a sense strand forming a double-stranded region, wherein said anti sense strand comprises 5′-AAGCAUUGUUAGCAGGAUUGC-3′ (SEQ ID NO:224), and said sense strand comprises 5′-AUCCUGCUAACAAUG-3′ (SEQ ID NO:434). The present invention also provides gene silencing agents comprising sense and antisense nucleotide sequences which are at least 90%, 91%, 92%, 93%, 94%, 95%, 96%, 97%, 98%, or 99% identical over their entire length to the foregoing sense and antisense nucleotide sequences.

In one aspect, the present invention provides gene silencing agents, wherein the gene silencing agent is double-stranded RNAi agent comprising an antisense strand and a sense strand forming a double-stranded region, wherein said anti sense strand comprises 5′-AACAAUUUGCGGCCAAUGUUU-3′ (SEQ ID NO:240), and said sense strand comprises 5′-CAUUGGCCGCAAAUU-3′ (SEQ ID NO:450). The present invention also provides gene silencing agents comprising sense and antisense nucleotide sequences which are at least 90%, 91%, 92%, 93%, 94%, 95%, 96%, 97%, 98%, or 99% identical over their entire length to the foregoing sense and antisense nucleotide sequences.

In one aspect, the present invention provides gene silencing agents wherein the gene silencing agent is double-stranded RNAi agent comprising an antisense strand and a sense strand forming a double-stranded region, wherein said anti sense strand comprises 5′-AAUUGUUUGGAGAAAUCAUCC-3′ (SEQ ID NO:245), and said sense strand comprises 5′-UGAUUUCUCCAAACA-3′ (SEQ ID NO:455). The present invention also provides gene silencing agents comprising sense and antisense nucleotide sequences which are at least 90%, 91%, 92%, 93%, 94%, 95%, 96%, 97%, 98%, or 99% identical over their entire length to the foregoing sense and antisense nucleotide sequences.

In one aspect, the present invention provides gene silencing agents, wherein the gene silencing agent is double-stranded RNAi agent comprising an antisense strand and a sense strand forming a double-stranded region, wherein said anti sense strand comprises 5′-AAACCAGCUACUUUAUCAUUG-3′ (SEQ ID NO:366), and said sense strand comprises 5′-UGAUAAAGUAGCUGG-3′ (SEQ ID NO:576). The present invention also provides gene silencing agents comprising sense and antisense nucleotide sequences which are at least 90%, 91%, 92%, 93%, 94%, 95%, 96%, 97%, 98%, or 99% identical over their entire length to the foregoing sense and antisense nucleotide sequences.

In one aspect, the present invention provides gene silencing agents, wherein the gene silencing agent is double-stranded RNAi agent comprising an antisense strand and a sense strand forming a double-stranded region, wherein said antisense strand comprises 5′-AAAUGUGUCACAAUUACCUUC-3′ (SEQ ID NO:354), and said sense strand comprises 5′-GGUAAUUGUGACACA-3′ (SEQ ID NO:564). The present invention also provides gene silencing agents comprising sense and antisense nucleotide sequences which are at least 90%, 91%, 92%, 93%, 94%, 95%, 96%, 97%, 98%, or 99% identical over their entire length to the foregoing sense and antisense nucleotide sequences.

In one aspect, the present invention provides gene silencing agents, wherein the gene silencing agent is double-stranded RNAi agent comprising an antisense strand and a sense strand forming a double-stranded region, wherein said antisense strand comprises 5′-AACAGCUGGACAAUCCUUAAG-3′ (SEQ ID NO:357), and said sense strand comprises 5′-AAGGAUUGUCCAGCU-3′ (SEQ ID NO:567). The present invention also provides gene silencing agents comprising sense and antisense nucleotide sequences which are at least 90%, 91%, 92%, 93%, 94%, 95%, 96%, 97%, 98%, or 99% identical over their entire length to the foregoing sense and antisense nucleotide sequences.

The gene silencing agents provided in any aspects above can be used for treating or preventing SARS-CoV-2 infection, inhibiting expression of SARS-CoV-2 protein in a cell, silencing SARS-CoV-2 gene in a cell, inhibiting replication of SARS-CoV-2 in a cell, or reducing SARS-CoV-2 titer in vitro or in vivo.

In another aspect, the present invention provides pharmaceutical compositions for treating conditions related to CoV infection, e.g., COVID-19, comprising gene silencing agents of any aspects provided by this invention and at least one pharmaceutically acceptable excipient or carrier. In some embodiments, the gene silencing agents is double-stranded RNAi agents comprising an antisense strand and a sense strand, wherein the antisense strand comprises at least 15 contiguous nucleotides differing by no more than 3 nucleotides from any one of the nucleotide sequences in FIGS. 1, 2 and 3 (SEQ ID NO: 211-420). In some embodiments, the gene silencing agents is double-stranded RNAi agents as provided in FIGS. 1-3 (GHI_N1 to GHI_N96, GHI_P97 to GHI_P162, aiRNA 3CL_1 to 28 and aiRNA PLPRO_1 to 20). In certain embodiments, the gene silencing agents is double-stranded RNAi agents selected from GHI_N43, GHI_P155, GHI_N14, GHI_N30, GHI_N35, GHI_P156, GHI_P144, GHI_P147, GHI_N31, GHI_N12, GHI_N3, GHI_N93, GHI_N13, GHI_N32, GHI_N29, GHI_N78, GHI_N33, GHI_N36, GHI_N91, GHI_N34, GHI_N20, GHI_N75, GHI_N95, GHI_N46, GHI_N15, GHI_N44, GHI_N96, GHI_N81, GHI_N58, GHI_N84, GHI_N86, GHI_N62, GHI_P157, GHI_P158, GHI_P151, GHI_P148, GHI_P145, GHI_P153, GHI_P150, GHI_P159, GHI_P146, GHI_P149, GHI_P154, GHI_P152, GHI_P160, GHI_P161 and GHI_P162.

In another aspect, the present invention provides pharmaceutical compositions for treating conditions related to CoV infection, e.g., COVID-19, wherein the pharmaceutical compositions comprise two or more gene silencing agents of any aspects provided by this invention and at least one pharmaceutically acceptable excipient or carrier, wherein each gene silencing agent is directed to a different segment of a CoV gene or to different CoV genes.

In some embodiments, the pharmaceutical compositions comprise two gene silencing agents and at least one pharmaceutically acceptable excipient or carrier, wherein the first gene silencing agent targets the Nucleocapsid gene of SARS-CoV-2, and the second gene silencing agent targets the RdRp gene of SARS-CoV-2. In some embodiments, the first gene silencing agent comprises an antisense strand consisting of, consisting essentially of or comprising at least 15 or more contiguous nucleotides differing by no more than 3 nucleotides from one of the antisense strand sequences provided in FIG. 1 (SEQ ID NOs: 211-306). In some embodiments, the second gene silencing agent comprises an antisense strand consisting of, consisting essentially of or comprising at least 15 or more contiguous nucleotides differing by no more than 3 nucleotides from one of the antisense strand sequences provided in FIG. 2 (SEQ ID NOs: 307-372). In some embodiments, the first gene silencing agent is a double-stranded RNAi agent selected from any one of GHI_N1 to GHI_N96. In some embodiments, the second gene silencing agent is a double-stranded RNAi agent selected from any one of GH I_P97 to GHI_P162.

In some embodiments, the pharmaceutical compositions comprise two gene silencing agents and at least one pharmaceutically acceptable excipient or carrier, wherein the first gene silencing agent targets the Nucleocapsid gene of SARS-CoV-2, comprising an antisense strand consisting of, consisting essentially of or comprising at least 15 or more contiguous nucleotides differing by no more than 3 nucleotides from one of the antisense strand sequences selected from the group consisting of SEQ ID NOs: 253, 224, 240 and 245, and the second gene silencing agent targets the RdRp gene of SARS-CoV-2, comprising an antisense strand consisting of, consisting essentially of or comprising at least 15 or more contiguous nucleotides differing by no more than 3 nucleotides from one of the antisense strand sequences selected from the group consisting of SEQ ID NOs: 365, 366, 354 and 357. In some embodiments, the first gene silencing agent comprises an antisense strand consisting of, consisting essentially of or comprising at least 15 or more contiguous nucleotides differing by no more than 3 nucleotides from the antisense strand sequences SEQ ID NO:253, and the second gene silencing agent comprises an antisense strand consisting of, consisting essentially of or comprising at least 15 or more contiguous nucleotides differing by no more than 3 nucleotides from the antisense strand sequences SEQ ID NO:365.

In some embodiments, the first gene silencing agent is a double-stranded RNAi agent selected from any one of the group consisting of GHI_N43, GHI_N14, GHI_N30, GHI_N35, GHI_N31, GHI_N12, GHI_N3, GHI_N93, GHI_N13, GHI_N32, GHI_N29, GHI_N78, GHI_N33, GHI_N36, GHI_N91, GHI_N34, GHI_N20, GHI_N75, GHI_N95, GHI_N46, GHI_N15, GHI_N44, GHI_N96, GHI_N81, GHI_N58, GHI_N84, GHI_N86 and GHI_N62. In some embodiments, the second gene silencing agent is a double-stranded RNAi agent selected from any one of the group consisting of GHI_P155, GHI_P156, GHI_P144, GHI_P147, GHI_P157, GHI_P158, GHI_P151, GHI_P148, GHI_P145, GHI_P153, GHI_P150, GHI_P159, GHI_P146, GHI_P149, GHI_P154, GHI_P152, GHI_P160, GHI_P161 and GHI_P162.

In one embodiment, the pharmaceutical compositions comprise two gene silencing agents, wherein the first gene silencing agent is an aiRNA molecule including an antisense strand and a sense strand forming a double-stranded region, wherein said antisense strand comprises at least 15 or more contiguous nucleotides differing by no more than 3 nucleotides from the antisense strand sequence SEQ ID NO:253, and said sense strand comprises at least 12 or more contiguous nucleotides differing by no more than 3 nucleotides from the sense strand sequence SEQ ID NO:463, and the second gene silencing agent is an aiRNA molecule including an antisense strand and a sense strand forming a double-stranded region, wherein said antisense strand comprises at least 15 or more contiguous nucleotides differing by no more than 3 nucleotides from the antisense strand sequence SEQ ID NO:365, and said sense strand comprises at least 12 or more contiguous nucleotides differing by no more than 3 nucleotides from the sense strand sequence SEQ ID NO:575.

Further, the gene silencing agents of any aspects provided by this invention can either contain only naturally occurring ribonucleotide subunits, or, can be synthesized to contain one or more modifications to the sugar or base of one or more of the ribonucleotide subunits that is included in the agent. In one embodiment, at least one of the nucleotides of the antisense strand and/or sense strand of the gene silencing agents provided in any aspects above are modified nucleotides. In one embodiment, substantially all of the nucleotides of the antisense strand and/or the sense strand of the gene silencing agents provided in any aspects above are modified nucleotides. The gene silencing agents can be further modified so as to be attached to one or more ligands, moieties or conjugates that is selected to improve stability, distribution or cellular uptake of the agent. The gene silencing agents can further be in isolated form or can be part of a pharmaceutical composition used for the methods described herein, particularly as a pharmaceutical composition formulated for delivery to the lungs or nasal passage or formulated for parenteral administration. The pharmaceutical compositions can contain one or more gene silencing agents, and in some embodiments, will contain two or more gene silencing agents, each one directed to a different segment of a CoV gene or to two different CoV genes.

The present invention further provides methods for reducing the level of CoV protein, especially SARS-CoV-2 protein, in a cell. The present invention further provides methods for treating or preventing CoV infection, especially SARS-CoV-2 infection, in a subject. Such methods comprise the step of administering at least one (e.g., one, two or more) of the gene silencing agents of the present invention to a subject. The present methods utilize the cellular mechanisms involved in RNA interference to selectively degrade the CoV protein level in a cell and are comprised of the step of contacting a cell with at least one (e.g., one, two or more) of the antiviral gene silencing agents of the present invention. Such methods can be performed directly on a cell or can be performed on a mammalian subject by administering to the subject at least one (e.g., one, two or more) of the gene silencing agents/pharmaceutical compositions of the present invention.

The details of one or more embodiments of the invention are set forth in the accompanying drawings and the description below. Other features, objects, and advantages of the invention will be apparent from this description, the drawings, and from the claims. This application incorporates all cited references, patents, and patent applications by references in their entirety for all purposes.

### BRIEF DESCRIPTION OF FIGURES

FIG. 1 illustrates exemplary sequences of rationally designed and selected target sequence of Nucleocapsid (N) region of SARS-CoV-2 gene along with the start nucleotide position of each target sequence, and rational designed antisense strand of the gene silencing agent, and rational designed sense strand of asymmetrical interfering RNA (aiRNA) corresponding to the designed antisense strand, along with the in vitro essay result of the relative expression.

FIG. 2 illustrates exemplary sequences of rationally designed and selected target sequence of RdRp region of SARS-CoV-2 gene along with the start nucleotide position of each target sequence, and rational designed antisense strand of the gene silencing agent, and rational designed sense strand of asymmetrical interfering RNA (aiRNA) corresponding to the designed antisense strand, along with the in vitro essay result of the relative expression.

FIG. 3 illustrates exemplary sequences of rationally designed and selected target sequence of 3CL and PL1 genes of SARS-CoV-2 genome, and rational designed antisense strand of the gene silencing agent, and rational designed sense strand of asymmetrical interfering RNA (aiRNA) corresponding to the designed antisense strand, along with the in vitro essay result of the relative expression.

FIG. 4 illustrates targeted gene regions of SARS-CoV-2, e.g., regions 5123-5929, 10052-10988, 13468-16237, 28374-29530 of GenBank accession #MN908947.3 were selected for gene silencing agents targeting.

FIG. 5 illustrates exemplary the entire SARS-CoV-2 genome and targeted RdRp and Nucleocapsid regions of SARS-CoV-2 genome for screening gene silencing agents.

FIG. 6 illustrates exemplary a general structure of 15mer duplex aiRNA, with 15mer-sense strand and 21mer antisense strand, having overhangs of 3 nucleotides at the 3′ and 5′ ends of the antisense strand.

FIG. 7 illustrates results complied from 96-well screenings for effective aiRNAs from SARS-CoV-2 candidate N targeting aiRNAs (GHI_N1 to GHI_N48) in HeLa cells at 1 nM. The ratios of Luc/Rluc were calculated against the mock-transfected control.

FIG. 8 illustrates results complied from 96-well screenings for effective aiRNAs from SARS-CoV-2 candidate N targeting aiRNAs (GHI_N49 to GHI_N96) in HeLa cells at 1 nM. The ratios of Luc/Rluc were calculated against the mock-transfected control.

FIG. 9 illustrates results complied from 96-well screenings for effective aiRNAs from SARS-CoV-2 N targeting super aiRNAs in HeLa cells at 100 pM. The ratios of Luc/Rluc were calculated against the mock-transfected control.

FIG. 10 illustrates results complied from 96-well screenings for effective aiRNAs from SARS-CoV-2 candidate RdRp targeting aiRNAs (GHI_P97 to GHI_P144) in HeLa cells at 1 nM. The ratios of Luc/Rluc were calculated against the mock-transfected control.

FIG. 11 illustrates further screening results complied from 96-well screenings for effective aiRNAs from SARS-CoV-2 RdRp targeting aiRNAs (GHI_P144 to GHI_P162) in HeLa cells at 100 pM and 10 pM. The ratios of Luc/Rluc were calculated against the mock-transfected control.

FIG. 12 illustrates SARS-Cov-2 viral infection assay results by dose-response curves and IC₅₀ for each tested compound and combination thereof. aiRNAs were added to cell-based SARS-Cov-2 infection system without using transfection.

### DETAILED DESCRIPTION OF THE INVENTION

### Definitions

As used herein, the singular form “a”, “an”, and “the” include plural references unless the context clearly dictates otherwise. For example, the term “a cell” includes a plurality of cells including mixtures thereof.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In some embodiments, the term “about” is used to modify a numerical value above and below the stated value by a variance of 10%. In some embodiments, the term “about” is used to modify a numerical value above and below the stated value by a variance of 5%. In some embodiments, the term “about” is used to modify a numerical value above and below the stated value by a variance of 1%.

As used herein, the term “nucleoside” means a compound comprising a nucleobase moiety and a sugar moiety. Nucleosides include, but are not limited to, naturally occurring nucleosides (e.g., deoxyribonucleosides and ribonucleosides as found in DNA and RNA) and modified nucleosides. Nucleosides may be linked to a phosphate moiety to become, for example, nucleotides.

As used herein, the term “nucleotide” means a nucleoside further comprising a phosphate linking group.

As used herein, the term “gene silencing agent”, comprising RNAi agent or ASO agent, is an unmodified RNA, modified RNA, or nucleoside surrogate, all of which are described herein or are well known in the RNA synthetic art. While numerous modified RNAs and nucleoside surrogates are described, preferred examples include those which have greater resistance to nuclease degradation than do unmodified RNAs. Preferred examples include those that have a 2′ sugar modification, a 5′-modification which includes one or more phosphate groups or one or more analogs of a phosphate group.

As used herein, the term “RNAi agent” is an RNA agent, which can down-regulate the expression of a target gene, e.g., CoV. While not wishing to be bound by theory, an RNAi agent may act by one or more of a number of mechanisms, including post-transcriptional cleavage of a target mRNA sometimes referred to in the art as RNAi, or pre-transcriptional or pre-translational mechanisms. In some embodiments, an RNAi agent is a double-stranded (ds) RNAi agent. In some embodiments, an RNAi agent is an antisense oligonucleotide.

As used herein, the term “ds RNAi agent” (abbreviation for “double-stranded RNAi agent”), as used herein, is an RNAi agent which includes more than one, and preferably two, strands in which interchain hybridization can form a region of duplex structure. A “strand” herein refers to a contiguous sequence of nucleotides (including non-naturally occurring or modified nucleotides). The two or more strands may be, or each form a part of, separate molecules, or they may be covalently interconnected, e.g., by a linker, e.g., a polyethyleneglycol linker, to form but one molecule. At least one strand can include a region which is sufficiently complementary to a target RNA. Such strand is termed the “antisense strand”. A second strand comprised in the ds RNAi agent which comprises a region complementary to the antisense strand is termed the “sense strand”. However, a double-stranded (ds) RNAi agent can also be formed from a single RNA molecule which is, at least partly; self-complementary, forming, e.g., a hairpin or panhandle structure, including a duplex region. In such case, the term “strand” refers to one of the regions of the RNA molecule that is complementary to another region of the same RNA molecule. “aiRNA agent” or “aiRNA” and “siRNA agent” or “siRNA” as used herein, refers to an iRNA agent, e.g., a ds RNAi agent, that is sufficiently short that it does not induce a deleterious interferon response in a human cell, e.g., it has a duplexed region of less than 30 nucleotide pairs. “aiRNA agent” or “aiRNA” has two strands that are asymmetric, namely either has overhangs of 1-5 nucleotides at both 3′ and/or 5′ ends of the antisense strand of the agent, or has one blunt end at the 5′ ends of the antisense strand and one overhang of 1-10 nucleotides at the 3′ ends of the antisense strand. “siRNA agent” or “siRNA” has two strands that are substantially symmetric, namely has two overhangs of 1-5 nucleotides at both 3′ ends of the agent. In a preferred embodiment, the ds RNAi agent is aiRNA (asymmetric interfering RNA) molecule(s).

As used herein, the term “oligonucleotide” refers to a compound comprising a plurality of linked nucleosides. In certain embodiments, one or more of the plurality of nucleosides is modified. In certain embodiment, an oligonucleotide comprises one or more ribonucleosides (as in RNA) and/or deoxyribonucleosides (as in DNA). In some embodiments, the oligonucleotide is a single-stranded oligonucleotide. In some other embodiments, the oligonucleotide is a double-stranded interfering RNA, such as siRNA, aiRNA, shRNA.

As used herein, the term “modified nucleotide” means a nucleotide having at least one modified sugar moiety, modified internucleoside linkage, and/or modified nucleobase.

As used herein, the term “modified nucleoside” means a nucleoside having at least one modified sugar moiety, and/or modified nucleobase.

As used herein, the term “modified oligonucleotide” means an oligonucleotide comprising at least one modified nucleotide.

As used herein, the term “naturally occurring internucleoside linkage” means a 3′ to 5′ phosphodiester linkage.

As used herein, the term “modified internucleoside linkage” refers to a substitution or any change from a naturally occurring internucleoside bond. For example, a phosphorothioate linkage is a modified internucleoside linkage.

As used herein, the term “natural sugar moiety” means a sugar found in DNA (2-H) or RNA (2-OH).

As used herein, the term “modified sugar” refers to a substitution or change from a natural sugar. For example, a 2′-O-methoxyethyl modified sugar is a modified sugar.

As used herein, the term “bicyclic sugar” means a furosyl ring modified by the bridging of two non-geminal ring atoms. A bicyclic sugar is a modified sugar.

As used herein, the term “modified nucleobase” refers to any nucleobase other than adenine, cytosine, guanine, thymidine, or uracil. For example, 5-methylcytosine is a modified nucleobase. An “unmodified nucleobase” means the purine bases adenine (A) and guanine (G), and the pyrimidine bases thymine (T), cytosine (C), and uracil (U).

As used herein, “modulating”, “regulating” and its grammatical equivalents refer to either increasing or decreasing (e.g., silencing), in other words, either up-regulating or down-regulating. As used herein, “gene silencing” refers to reduction of gene expression and may refer to a reduction of gene expression about 20%, 30%, 40%, 50%, 60%, 70%, 80%, 90% or 95% of the targeted gene.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

Terms such as “treating” or “treatment” or “to treat” or “alleviating” or “to alleviate” as used herein refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus, those in need of treatment include those already with the disorder; those prone to have the disorder; and those in whom the disorder is to be prevented. A subject is successfully “treated” according to the methods of the present invention if the patient shows one or more of the following: a reduction in the number of or complete absence of cancer cells; a reduction in the tumor size; inhibition of or an absence of cancer cell infiltration into peripheral organs including the spread of cancer into soft tissue and bone; inhibition of or an absence of tumor metastasis; inhibition or an absence of tumor growth; relief of one or more symptoms associated with the specific cancer; reduced morbidity and mortality; and improvement in quality of life.

As used herein, the terms “inhibiting”, “to inhibit” and their grammatical equivalents, when used in the context of a bioactivity, refer to a down-regulation of the bioactivity, which may reduce or eliminate the targeted function, such as the production of a viral protein or a viral mRNA. In particular embodiments, inhibition may refer to a reduction of about 20%, 30%, 40%, 50%, 60%, 70%, 80%, 90% or 95% of the targeted expression.

As used herein, the term “substantially complementary” or “complementary” refers to complementarity in a base-paired, double-stranded region between two nucleic acids and not any single-stranded region such as a terminal overhang or a gap region between two double-stranded regions. The complementarity does not need to be perfect; there may be any number of base pair mismatches, for example, between the two nucleic acids. However, if the number of mismatches is so great that no hybridization can occur under even the least stringent hybridization conditions, the sequence is not a substantially complementary sequence. When two sequences are referred to as “substantially complementary” herein, it means that the sequences are sufficiently complementary to each other to hybridize under the selected reaction conditions. The relationship of nucleic acid complementarity and stringency of hybridization sufficient to achieve specificity is well known in the art. Two substantially complementary strands can be, for example, perfectly complementary or can contain from 1 to many mismatches so long as the hybridization conditions are sufficient to allow, for example discrimination between a pairing sequence and a non-pairing sequence. Accordingly, substantially complementary sequences can refer to sequences with base-pair complementarity of at least, 70%, 75%, 80%, 85%, 86%, 87%, 88%, 89%, 90%, 91%, 92%, 93%, 94%, 95%, 96%, 97%, 98%, 99%, or 100%, or any number in between, in a double-stranded region.

As used herein, “fully complementary” or “100% complementary” means each nucleobase of a nucleobase sequence of a first nucleic acid has a complementary nucleobase in a second nucleobase sequence of a second nucleic acid. In certain embodiments, a first nucleic acid is an antisense strand and a second nucleic acid is a target nucleic acid. In certain embodiments, a first nucleic acid is a sense strand and a second nucleic acid is an antisense strand.

As used herein, “essentially identical” when used referring to a first nucleotide sequence in comparison to a second nucleotide sequence means that the first nucleotide sequence is identical to the second nucleotide sequence except for up to one, two or three nucleotide substitutions (e.g., adenosine replaced by uracil).

As used herein, “hybridization” means the annealing of complementary nucleic acid molecules. In certain embodiments, complementary nucleic acid molecules include a sense strand and an antisense strand. In certain embodiments, complementary nucleic acid molecules include an antisense strand and a target nucleic acid.

As used herein, “N gene” means the Nucleocapsid region of CoV genome as target gene, preferred example is the Nucleocapsid region of SARS CoV-2 genome, namely, region 28374-29530 of GenBank accession #MN908947.3. Nucleocapsid plays an important role in CoV assembly.

As used herein, “RdRp gene”, “POL gene” or “P gene” means the RdRp (RNA dependent RNA polymerase) region of CoV genome as target gene, preferred example is the RdRp region of SARS CoV-2 genome, which is known as the Non-structural protein 12 (Nsp12) of SARS CoV-2 genome, namely region 13468-16237 of GenBank accession #MN908947.3. RdRp plays an important role in CoV replications.

As used herein, “3CL gene” means the 3C-like protease region of CoV genome as target gene, preferred example is the 3CL region of SARS CoV-2 genome, namely, region 10052-10988 of GenBank accession #MN908947.3.

As used herein, “PL1 gene” means the Papain-like 1 protease region of CoV genome as target gene, preferred example is the PL1 region of SARS CoV-2 genome, namely, region 5123-5929 of GenBank accession #MN908947.3.

The terms “administer,” “administering,” or “administration” are used herein in their broadest sense. These terms refer to any method of introducing to a subject a compound or pharmaceutical composition described herein and can include, for example, introducing the compound systemically, locally, or in situ to the subject. Thus, a compound of the present disclosure produced in a subject from a composition (whether or not it includes the compound) is encompassed in these terms. When these terms are used in connection with the term “systemic” or “systemically,” they generally refer to in vivo systemic absorption or accumulation of the compound or composition in the blood stream followed by distribution throughout the entire body.

The terms “effective amount” and “therapeutically effective amount” refer to that amount of a compound or pharmaceutical composition described herein that is sufficient to affect the intended result including, but not limited to, disease treatment, as illustrated below. In some embodiments, the “therapeutically effective amount” is the amount that is effective for detectable killing or inhibition of the growth or spread of cancer cells, the size or number of tumors, and/or other measure of the level, stage, progression and/or severity of the cancer. In some embodiments, the “therapeutically effective amount” refers to the amount that is administered systemically, locally, or in situ (e.g., the amount of compound that is produced in situ in a subject). The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of cell migration. The specific dose may vary depending on, for example, the particular pharmaceutical composition, subject and their age and existing health conditions or risk for health conditions, the dosing regimen to be followed, the severity of the disease, whether it is administered in combination with other agents, timing of administration, the tissue to which it is administered, and the physical delivery system in which it is carried.

The term “pharmaceutical composition” is a formulation containing the disclosed Gene Silencing Agent in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient in a unit dose of composition is an effect the amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, intranasal, and the like. Dosage forms for the topical or transdermal administration of a silencing agent of this invention include powders, sprays, ointments, pastes, creams, lotions, gels, solutions, patches and inhalants.

### SARS-CoV-2 and its Genome

SARS-CoV-2 is a SARS-like corona virus. The closest virus to SARS-CoV-2 identified so far is a virus that originated from the Rhinolophus bat, showing >96% sequence homology (Fisher and Heymann, 2020). SARS-CoV-2 is only 79% homologous with the original SARS-CoV (Zhou et al, 2020).

The genome of SARS coronaviruses consists of a single-stranded, positive-sense RNA molecule approximately 30 kb in length (Marra et al., 2003). The large SARS-CoV RNA genome produces eight 3′-co-terminal, nested subgenomic mRNAs (sg-mRNAs) for the efficient translation of structural and accessory proteins (Masters, 2006). The 5′ two-thirds of the SARS-CoV genome encode two large replicase polyproteins, expressed by open reading frames (ORF) 1a and 1b. As in other coronaviruses, ORF1a and ORF1b are slightly overlapped and, because ORF1b lacks its own translation initiation sites, proteins encoded by ORF1b are only translated as a fusion protein together with ORF1a by programmed −1 ribosomal frameshifting (−1 PRF; Baranov et al., 2005). The ORF1a and ORF1a/1b fusion proteins are proteolytically cleaved into 16 mature nonstructural proteins (nsps) that play multiple crucial roles during viral genome replication (Masters, 2006). The −1 PRF is thought to be essential for CoV genome replication because the coronavirus RNA-dependent RNA polymerase (RdRp), the key component of the replicase required for viral genome replication (Xu et al., 2003), is the first part of the ORF1a/1b protein synthesized after frameshifting.

CoVs enter the host cell using the endosomal pathway and/or the cell surface non-endosomal pathway. CoVs disassemble inside the host cell and release the nucleocapsid and viral RNA into the cytoplasm, after which ORF1a/b is translated into polyprotein 1a (pp1a) and 1ab (pp1ab), and the genomic RNA is replicated. (Chan et al, 2015). Subgenomic mRNAs are then synthesized and translated to produce the structural and accessory proteins (van Boheemen, 2012). The helical nucleocapsid, formed by the assembly of nucleocapsid protein (N) and genomic RNA, then interacts with surface protein (S), envelope protein (E), and membrane protein (M) to form the assembled virion (Chan et al, 2015). The virion is eventually released into the extracellular compartment by exocytosis and the viral replication cycle is repeated (Chan et al, 2015).

### Design and Selection of Gene Silencing Agent

The present invention is based on the demonstration of target gene silencing of a SARS-CoV-2 gene in vitro. Based on these results, the invention specifically provides a gene silencing agent that can be used in treating viral infection, particularly CoVs and in particular SARS-CoV-2 infection, in isolated form and as a pharmaceutical composition.

The gene silencing agents of the present invention have been designed to target regions in the CoV genome, particular SARS-CoV-2 genome, that are most conserved domains to avoid, as much as possible, potential mutations happening in the target site. In addition, the gene silencing agents of the present invention have been designed to inhibit the crucial step of the CoV life cycle, e.g., assembly and replication. Recent Covid19 viral investigations indicate that Nucleocapsid (N) and RdRp (P) regions have a lower mutation rate than others such as the S protein. In some embodiments, the gene silencing agents of the present invention have been designed to target Nucleocapsid (N) and RdRp regions in SARS-CoV-2.

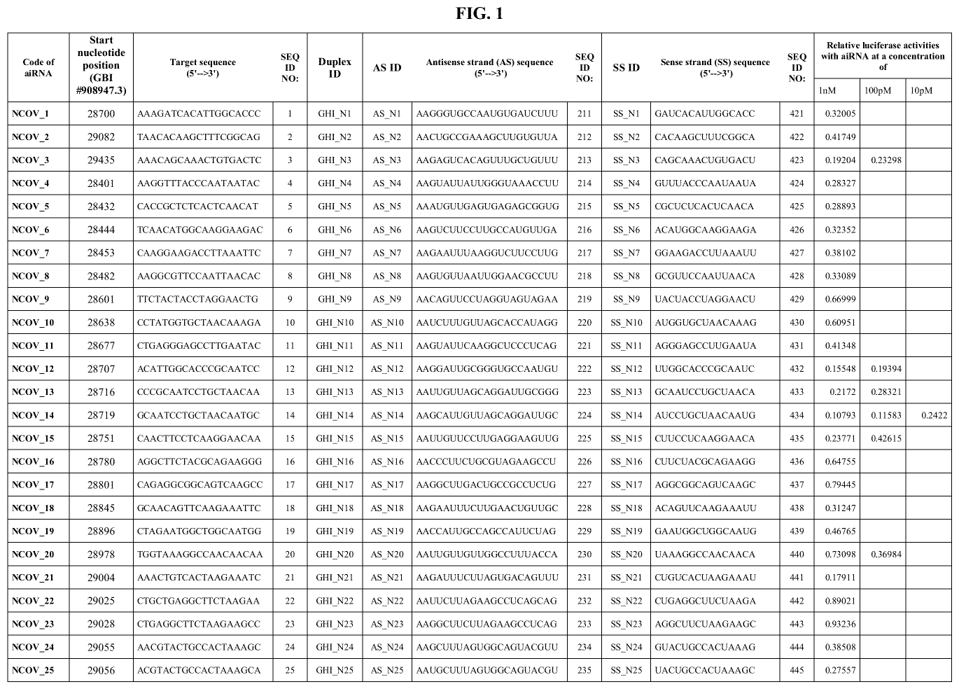

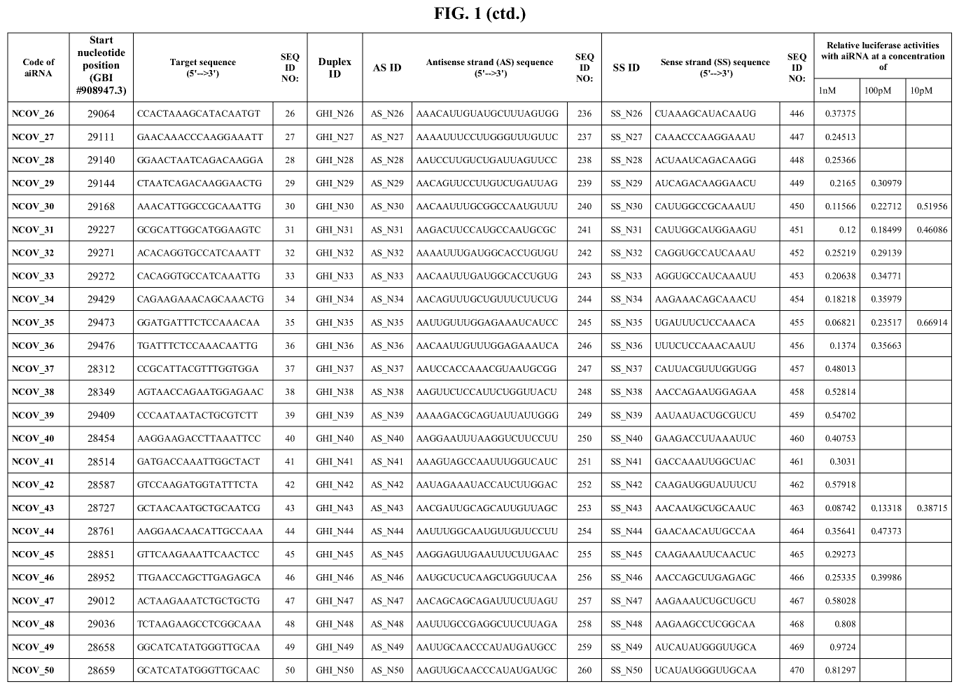

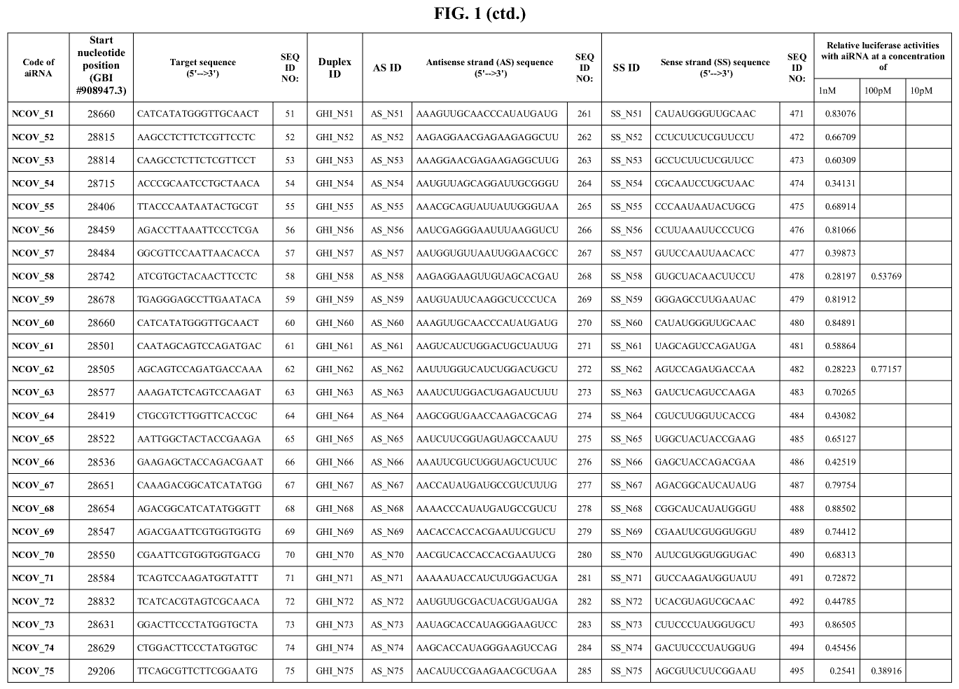

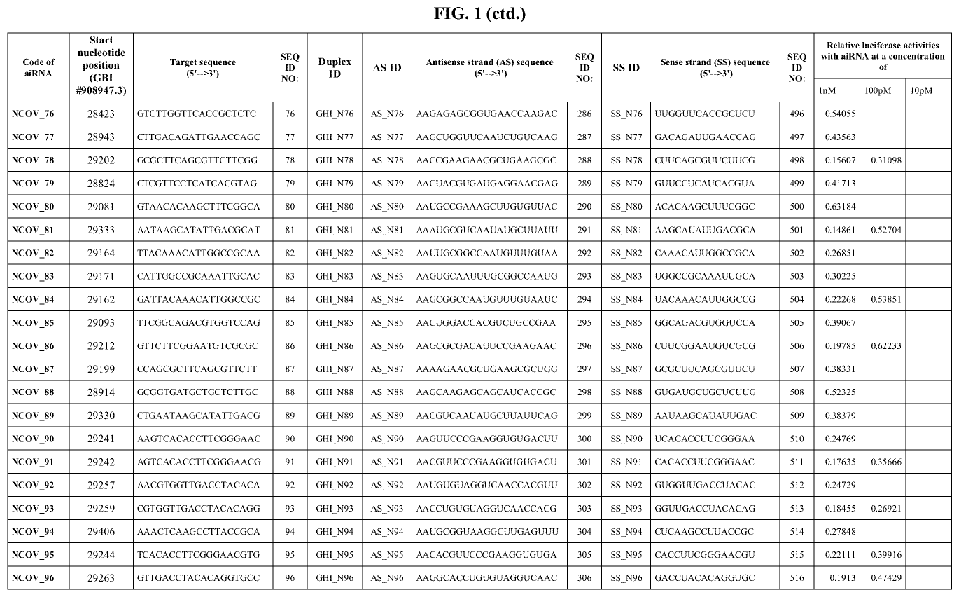

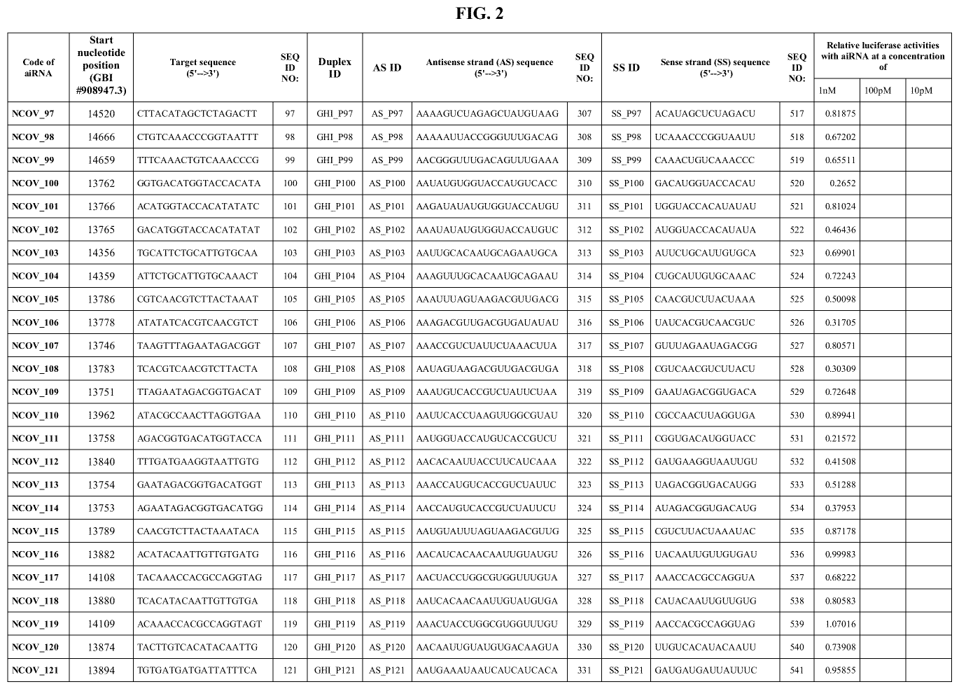

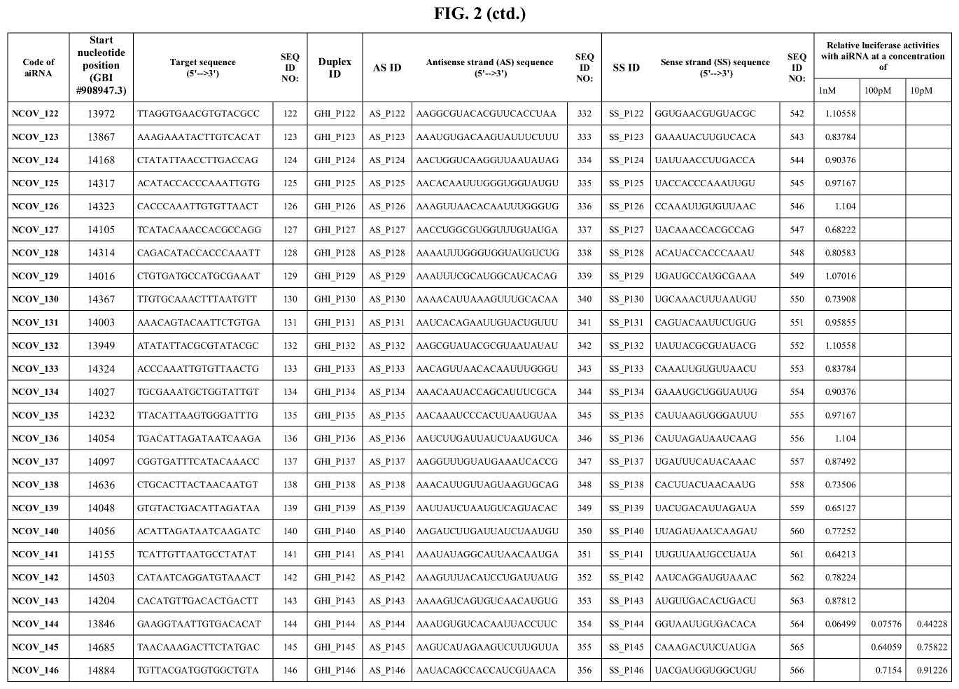

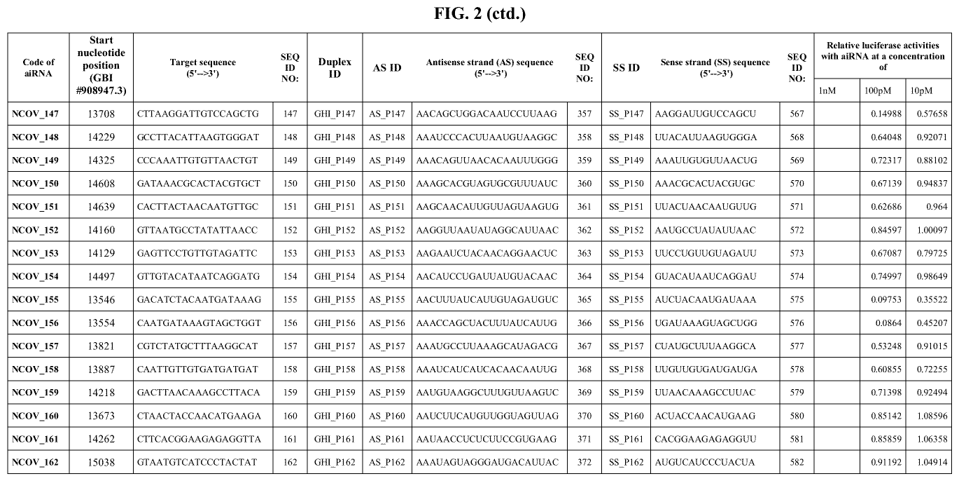

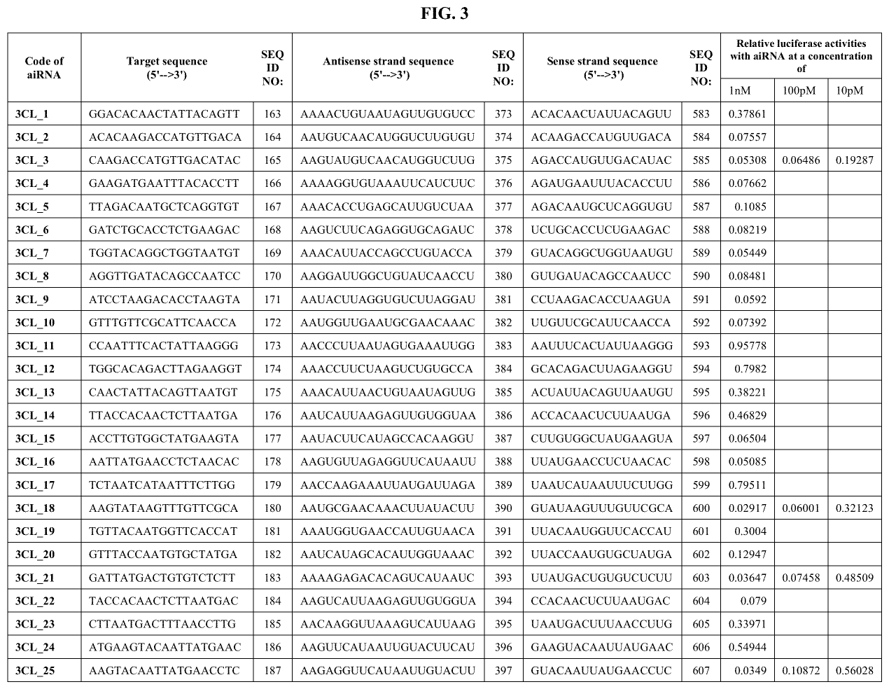

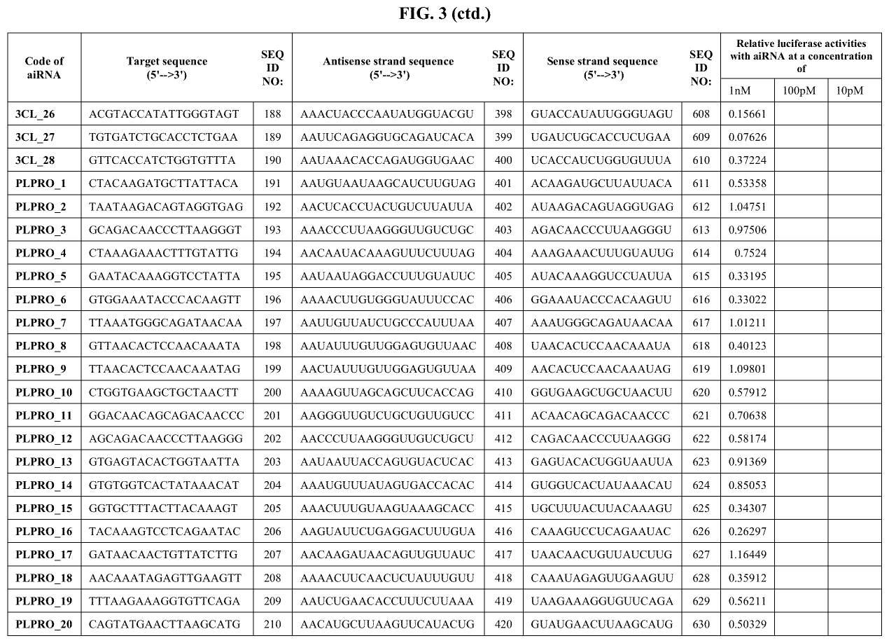

Such agents can be a single-stranded oligonucleotide or a double-stranded (ds) RNAi agent comprising an antisense strand having at least 15 or more contiguous nucleotides that are substantially complementary to a SARS-CoV-2 gene sequence, particularly, the PL1, 3CL, RdRp and N gene sequences of SARS-CoV-2, and more specifically, target sequences provided in FIGS. 1-3 (SEQ ID NOS. 1-210).

The present invention provides gene silencing agent comprising an antisense strand comprising a sequence of at least 9, 10, 11, 12, 13, 14, 15, 16, 17, 18, 19, 20, 21, 22, 23, 24, 25, 26, 27, 28, 29 or 30 nucleotides which is substantially complementary to, as defined above, at least a portion of a gene from a CoV, particularly the PL1, 3CL, RdRp and N gene of SARS-CoV-2. Exemplified antisense strand of the gene silencing agent, including ASO, siRNA and aiRNA, comprise 15 or more contiguous nucleotides differing by no more than 3 nucleotides from one of the designed antisense strands provided in FIGS. 1-3 (SEQ ID No. 211-420).

The gene silencing agent is a ds RNAi agent comprising an antisense strand having at least 15 or more contiguous nucleotides that are substantially complementary to a SARS-CoV-2 gene sequence, and a sense strand having at least 12 or more contiguous nucleotides that are substantially complementary to the antisense strand sequence. Particularly useful agents are ds RNAi agents comprising an antisense strand that consist of, consist essentially of or comprise a nucleotide sequence substantially complementary to a sequence from the PL1, 3CL, RdRp and N gene of SARS-CoV-2, more specifically, target sequences provided in FIGS. 1-3.

The antisense strands of the ds RNAi agents of the present invention are based on and comprise at least 15 or more contiguous nucleotides from one of the ds RNAi agents shown to be active in FIGS. 1-3. In such agents, the antisense strand of the agent can consist of, consist essentially of or comprise the entire antisense strand sequence provided in FIGS. 1-3 or can comprise 15 or more contiguous residues provided in FIGS. 1-3 along with additional nucleotides complementary with contiguous regions of the target gene. A ds RNAi agent can be rationally designed based on sequence information and desired characteristics and the information provided in FIGS. 1-3. For example, ads RNAi agent can be designed according to sequence of the agents provided in FIGS. 1-3 as well as in view of the entire coding sequence of the target gene.

Accordingly, the present invention provides ds RNAi agents comprising a sense strand and an antisense strand each comprising a sequence of at least 9, 10, 11, 12, 13, 14, 15, 16, 17, 18, 19, 20 or 21 nucleotides targeting a gene from a CoV, particularly the PL1, 3CL, RdRp and N genes of SARS-CoV-2. Exemplified ds RNAi agents include those that comprise 15 or more contiguous nucleotides from one of the agents provided in FIGS. 1-3.

In certain embodiments, the antisense strand of the gene silencing agent is 10 to 30 nucleotides in length. In other words, antisense strands are from 10 to 30 linked nucleobases. In other embodiments, the antisense strand comprises a modified oligonucleotide consisting of 8 to 100, 10 to 80, 10 to 50, 10 to 30, 12 to 50, 12 to 30, 14 to 30, 14 to 27, 15 to 23, 16 to 23, 19 to 23, or 21 linked nucleobases. In certain such embodiments, the antisense strand comprises a modified oligonucleotide consisting of 8, 9, 10, 11, 12, 13, 14, 15, 16, 17, 18, 19, 20, 21, 22, 23, 24, 25, 26, 27, 28, 29 or 30 linked nucleobases in length, or a range defined by any two of the above values.

In certain embodiments, the sense strand of the gene silencing agent is 9 to 30 nucleotides in length. In other words, sense strands are from 9 to 30 linked nucleobases. In other embodiments, the sense strand comprises a modified oligonucleotide consisting of 8 to 100, 10 to 80, 10 to 50, 10 to 30, 12 to 50, 12 to 30, 12 to 20, 12 to 17, 14 to 30, 14 to 20, 14 to 17, 15 to 23, 15 to 16, or 15 linked nucleobases. In certain such embodiments, the sense strand comprises a modified oligonucleotide consisting of 8, 9, 10, 11, 12, 13, 14, 15, 16, 17, 18, 19, 20, 21, 22, 23, 24, 25, 26, 27, 28, 29 or 30 linked nucleobases in length, or a range defined by any two of the above values.

In certain embodiments, the ds RNAi agent is an aiRNA molecule. Typically, the aiRNA agent comprises a short RNA duplex having a shorter sense strand and a longer antisense strand, wherein the duplex contains overhangs at the 3′ and/or 5′ ends of the antisense strand. In certain embodiments, the antisense strand has a 3′-overhang from 1, 2, 3, 4, 5, 6, 7 or 8 nucleotides and a 5′-overhang from 1, 2, 3, 4, 5, 6, 7 or 8 nucleotides. In another embodiment, the antisense strand has a 3′-overhang from 1, 2, 3, 4, 5, 6, 7, 8, 9 or 10 nucleotides and a 5′ blunt end. In yet another embodiment, the antisense strand has a 5′-overhang from 1, 2, 3, 4, 5, 6, 7, 8, 9 or 10 nucleotides and a 3′ blunt end.

In certain configurations, the aiRNA comprises modified nucleotides. The modified aiRNA is more resistant to degradation and less likely to elicit an immune response compared to a corresponding unmodified aiRNA sequence; while retaining RNAi activity against the target gene. Further, such modifications allow gene silencing at therapeutically viable aiRNA doses with very low cytokine induction, toxicity, and off-target effects associated with the use of unmodified aiRNA. In some configurations, the modified aiRNA contains at least one 2′-O-methyl (2′OMe) purine or pyrimidine nucleotide such as a 2′OMe-uridine, 2′OMe-adenosine, 2′OMe-guanosine, and/or 2′OMe-cytosine nucleotide. In some configurations, the modified aiRNA contains at least one 2′-deoxy-2′-fluoro nucleotide. The modified nucleotides can be present in one strand (i.e., sense or antisense) or both strands of the aiRNA.

In certain configuration, aiRNA duplexes of various lengths (e.g., 9-23, 14-21, 15-19, 15-21, 14-19, or 14-20 base pairs, more typically 9, 10, 11, 12, 13, 14, 15, 16, 17, 18, 19, 20, 21, 22 or 23 base pairs) may be designed with overhangs at the 3′ and/or 5′ ends of the antisense strand. In certain instances, the sense strand of the aiRNA molecule is about 9-23, 14-21, 15-19, 15-21, 14-19, or 14-20 nucleotides in length, more typically 9, 10, 11, 12, 13, 14, 15, 16, 17, 18, 19, 20, or 21 nucleotides in length. In certain other instances, the antisense strand of the aiRNA molecule is about 15-50, 15-40, or 15-30 nucleotides in length, more typically about 15, 16, 17, 18, 19, 20, 21, 22, 23, 24 or 25, or 19, 20, 21, 22 or 23 nucleotides in length, and preferably about 19-23 nucleotides in length.

In certain configuration, the present invention provides designed aiRNA duplexes contain 15mer sense strand (ss) and 21mer antisense strand (as), targeting four regions of the SARS-CoV-2 genome including the Nucleocapsid region (28374-29530), RdRp (P) region (13468-16237), 3CL region (10052-10988) and PL1 region (5123-5929), particularly, targeting the Nucleocapsid (N) region and RdRp region.

In some configurations, the 5′ antisense overhang contains one, two, three, or more nontargeting nucleotides (e.g., AA, UU, dTdT, etc.). In other configurations, the 3′ antisense overhang contains one, two, three, or more nontargeting nucleotides (e.g., AA, dTdT, etc.). In certain configurations, the 5′ terminal nucleotide and the first nucleotide adjacent to the 5′ terminal nucleotide of the antisense strand is an “AA” motif, a “UU” motif, a “CC” motif, an “AU” motif, an “AC” motif, a “UA” motif, a “UC” motif, or a “CA” motif, or a “dTdT” motif. In a prefer configuration, the 5′ terminal nucleotide and the first nucleotide adjacent to the 5′ terminal nucleotide of the antisense strand comprise an “AA” motif. In some configurations, the last nucleotide of antisense strand at 3′ end consists of an A, U, G or C ribonucleotide.

In some configurations, the gene silencing agent molecules may comprise one or more modified nucleotides, e.g., in the double-stranded region and/or in the single-stranded region. Typically, these modifications include deoxyribonucleosides, 2′OMe nucleotides (such as, for example, 2′OMe-uridine nucleotides, 2′OMe-adenosine nucleotides, 2′OMe-cytosine nucleotides, or 2′OMe-guanosine nucleotides.), 2′-fluorine nucleotides (2′-deoxy-2′-fluoro nucleotides), 2;-MOE nucleotides, LNA, cEt Morpholine and/or a phosphorothioate (P═S), phosphodiester (P═O) or Thio-phosphoramidate internucleoside linkage, and/or a 5′-Me C nucleotides.

In some configurations, the gene silencing agents can be further modified so as to be attached to one or more ligands, moieties or conjugates that is selected to improve stability, distribution or cellular uptake of the agent. Such moieties include but are not limited to lipid moieties such as a cholesterol moiety, cholic acid, a thioether, e.g., beryl-S-tritylthiol, a thiocholesterol, an aliphatic chain, e.g., dodecandiol or undecyl residues, a phospholipid, e.g., di-hexadecyl-rac-glycerol or triethyl-ammonium 1,2-di-O-hexadecyl-rac-glycero-3-phosphonate, a polyamine or a polyethylene glycol chain, or adamantane acetic acid, a palmityl moiety, or an octadecylamine or hexylamino-carbonyloxycholesterol moiety. In one embodiment, a ligand alters the distribution, targeting or lifetime of a gene silencing agent into which it is incorporated. In preferred embodiments, a ligand provides an enhanced affinity for a selected target, e.g., molecule, cell or cell type, compartment, e.g., a cellular or organ compartment, tissue, organ or region of the body, as, e.g., compared to a species absent such a ligand. Preferred ligands will not take part in duplex pairing in a duplexed nucleic acid. Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylgalactosamine, or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide. In some configurations, the ligands, moieties or conjugates can be conjugated to the 3′ terminus of the sense strand through any linker known to one skilled in the art. In some configurations, the ligands, moieties or conjugates can be conjugated to the 5′ terminus of the sense strand through any linker known to one skilled in the art. In some configurations, the ligands, moieties or conjugates can be conjugated to the 3′ terminus of the antisense strand through any such linker. In some configurations, the ligands, moieties or conjugates can be conjugated to the 5′ terminus of the antisense strand through any such linker.

In some configurations, the anti sense strand and/or the sense strand contains an overall GC content of 20-70%. In a further embodiment, the GC content is less than 50%, or preferably 30-50%.

Suitable aiRNA/siRNA/ASO sequences can be designed, selected, synthesized, and modified using any means known in the art.

a) Design and Selection of aiRNA Sequences

Lead aiRNA sequences were generated and ranked through a custom bioinformatics-based approach. A list of all possible aiRNA target sequences was generated by splitting the SARS-CoV-2 mRNA transcript sequences found in NCBI into all possible 19 base pair target sequences. Regions of sequence divergence were then removed, to avoid targeting polymorphic sequences. Excessively high GC content in a sequence can lead to difficulty unwinding due to high thermodynamic stability whereas excessively low GC content can lead to reduced binding to target sequences. As such, sequences with excessively high or low GC Content, as defined by >70% or <25% respectively, were removed. (GC Content is calculated as the percentage of guanine and cytosine bases in the target sequence). Sequences with four or more consecutive identical bases (e.g., AAAA, TTTT, CCCC, GGGG) were also removed as these were unlikely to properly anneal. The remaining sequences were screened for their ability to silence the expression of the gene of interest (GOI), beginning with sequences with the highest likelihood of efficacy as determined by the presence of patterns associated with previously found high efficacy sequences.

In certain embodiments, the gene silencing agent comprises an antisense strand consisting of, consisting essentially of or comprising a sequence same with at least 15 contiguous nucleotides differing by no more than 3 nucleotides from one of the sequences of SEQ ID NOS: 211 to 420. In certain embodiments, the antisense strand consists of, consists essentially of or comprises a sequence same with at least 19 contiguous nucleotides differing by no more than 3 nucleotides from one of the sequences of SEQ ID NO.: 211 to 420. In certain embodiments, the antisense strand consists of, consists essentially of or comprises a sequence selected from SEQ ID NO.: 211 to 420.

b) Generating aiRNA Molecules

aiRNA are typically generated via solid-phase chemical synthesis using the phosphoramidite method (Beaucage et al., 1981). However, the individual oligonucleotides that make up the aiRNA molecules can be synthesized by any variety of techniques described in the literature for oligonucleotide synthesis, including liquid-phase or solid-phase phosphodiester, phosphotriester, phosphite triester, or H-phosphonate chemistry (Reese et al., 2005). Standard oligonucleotide synthesis makes use of common nucleic acid protecting groups, such as dimethoxytrityl at the 5′ end and benzoyl, isobutyryl or acetyl on heterocyclic bases. Furthermore, aiRNA synthesis for applications in vivo often incorporates chemical modifications that improve serum stability and reduce off-target effects, such as 2′-O-methyl and 2′-fluoro modifications to the sugar backbone (Behlke 2009). Computer-controlled, automated oligonucleotide synthesizers such as the Mermade series from Bioautomation (Irving, Tex.) can be used for high-throughput production of aiRNA molecules (Rayner et al. 1998). aiRNA can be synthesized on these and similar instruments at a 0.2 μmol or 1.0 μmol scale, although larger and smaller scale syntheses are also possible. Suitable reagents for RNA synthesis, deprotection and purification are commercially available from a variety of manufacturers. Standard protocols for these processes are readily available in the literature and/or direct from the manufacturer.

### Evaluation of Candidate RNAi Agents

Screening of SARS-CoV-2 aiRNAs

Not all sequences within a specific messenger RNA are suitable targets for RNA interference. For instance, it is well known that secondary structures in the messenger RNA molecule can prevent the hybridization of the anti-sense strand, an essential step for RNA interference. A proprietary algorithm was employed to guide the selection of aiRNAs, see section “Design and selection of aiRNA sequences”. Subsequently, the efficacy of aiRNAs was verified in vitro assays.

To test the efficacy of aiRNAs in vitro, aiRNAs can be delivered to cells expressing the GOI. Methods well known in the art, such as encapsulation of aiRNAs in liposomes that are then taken up by the cells, can be used to deliver the aiRNAs. Subsequently, one can assess the efficacy with which a specific aiRNA suppresses the GOI by comparing the expression level of the GOI in cells that received empty liposomes, or an unspecific aiRNA (e.g., aiRNA that does not target the GOD, with the expression of the GOI in cells that received the specific aiRNA. The expression of the GOI can be studied on the RNA as well as the protein level, using techniques well known in the art, such as quantitative PCR for RNA, and Immunoblot or ELISA for protein.

Synthesis of Duplex aiRNAs

A proprietary algorithm to guide the selection of aiRNAs from the predicted mRNA sequence of SARS-CoV-2 is employed. Selected candidate aiRNAs are showed (See FIG. 1 to FIG. 3). The candidate aiRNAs (15mer SS and 21mer AS) were synthesized using phosphoramidite chemistry by bioautomation synthesizer at 1Globe Health Institute, Norwood. The aiRNA duplexes were prepared following the standard annealed protocols. aiRNA annealing was confirmed by polyacrylamide gel electrophoresis.

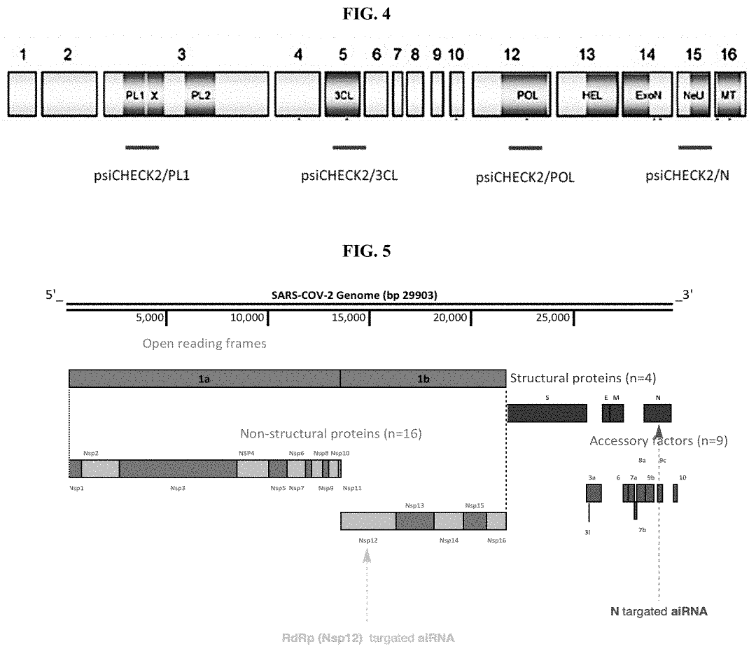

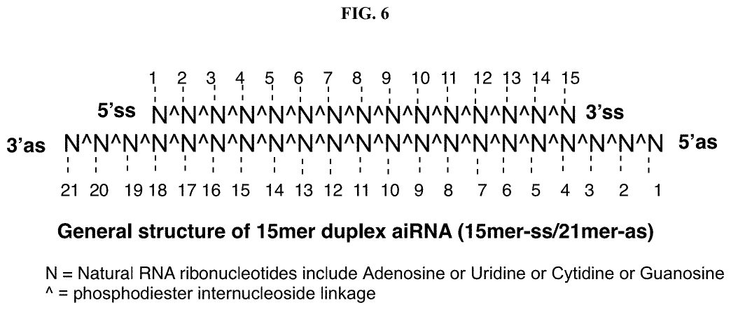

To identify aiRNAs that efficiently suppress expression of SARS CoV-2 genes, we subcloned the relevant viral sequences (based on the known SARS CoV-2 genome; FIG. 4 and FIG. 5) in a standard reporter vector that expresses the firefly luciferase gene. Then we screened hundreds of aiRNAs by delivering both the relevant recombinant vector and each aiRNA to mammalian cells and then comparing the expression of the reporter (luciferase) in cells that had received a specific aiRNA to reporter expression in control cells (mock-transfected cells). aiRNAs were screened at different concentrations, and the most effective ones were chosen for determination of IC₅₀ concentrations. A list of the aiRNAs that were screened and the screening results, is given in FIG. 1 to FIG. 3 and FIG. 7 to FIG. 11.

### Example 1: In Vitro Screening of all Candidate aiRNAs that Silence SARS-CoV-2 Gene Expression in Mammalian Cells

The Dual-Luciferase COVID-19 reporter (DLR) plasmids psiCHECK2/PL1, psiCHECK2/3CL, psiCHECK2/RdRp (P) and psiCHECK2/Nucleocapsid (N), containing a synthetic version of the Renilla luciferse (hRluc) gene and a synthetic firefly luciferase (hluc), were constructed to test the activity of COVID-19 aiRNAs. SARS-CoV-2 sequence regions PL1 (5123-5929) 3CL (10052-10988), RdRp (13468-16237), and N (28374-29530) (See FIG. 4) of GenBank accession #MN908947.3 were joined in, restriction sites were added at both ends, and this sequence was then chemically synthesized and subcloned into the psiCHECK2 Dual-Luciferase vector.

HeLa cells were grown in DMEM medium, supplemented with fetal calf serum, penicillin, and streptomycin. Sense and antisense aiRNA strands were chemically synthesized on a Mermade 192 oligonucleotide synthesizer. HeLa cells were plated at 10000 cells/well into 96-well culture plates. 24 hours later, these cells were co-transfected with reporter plasmid DNA and various concentrations of COVID-19 aiRNAs (10 pM, 100 pM, 1 nM), unspecific siRNA (negative control) or mock-transfected, using Lipofectamine 2000 reagent. 24 hours post-transfection, the effect of each aiRNA on COVID-19 gene expression was determined by Dual-Glo Luciferase Assay System. Screening results are shown in FIG. 1 to FIG. 3.

### Example 2: In Vitro Screening of N Targeting aiRNAs (GHI_N1 to GHI_N96) and RdRp Targeting aiRNAs (GHI_P97 to GHI_P162) in Mammalian Cells

Construction of Dual-Luciferase Reporter (DLR) system

Two DLR reporter plasmids including psiCheck-2/Nucleocapsid (N) and psiCheck-2/RdRp (P) were constructed. In brief, from Genbank accession number MN908947.3 (SARS-COV-2 isolate Wuhan-Hu-1), the target regions RdRp (13468-16237) and N (28374-29530) were chemically synthesized by Integrated DNA Technologies (IDT) and were amplified by PCR. Restrictions sites for XhoI and NotI were added to forward and reverse primers, respectively. The PCR amplified P and N fragments were cloned into the psiCheck-2 vector (Promega), in which two luciferase genes (Firefly and Renilla) driven by separate promoters are present. Targeting fragment inserts were cloned into a region immediately following the Renilla luciferase gene, which serves as the reporter, while the Firefly luciferase gene is internal control.

Cell culture and Transfection

Anti-SARS-CoV-2 activities of aiRNAs were studied in human cervical cancer cell line HeLa. The cells were cultured in Dulbecco's modified Eagle's medium-1X (DMEM, Gibco) supplemented with 10% heat-inactivated fetal bovine serum (FBS), 100 U/ml penicillin, and 100 μg/ml streptomycin at 37° C. in a humidified atmosphere of 5% CO₂.

For 96 wells, HeLa cells were seeded in 100 uL DMEM media without antibiotics to reach 70%-80% confluence at the time of transfection. The reporter plasmid psiCheck-2/N or psiCheck-2/P and aiRNA-cholesterol conjugates were co-transfected using Lipofectamine 2000 (Invitrogen) in the OptiMEM-I reduced-serum media (Invitrogen). Twenty-four hours after transfection, the luminescence activity of the Firefly-luciferase and Renilla-luciferase in each well was detected by using the standard Dual-Luciferase Reporter kit protocol (Promega standard DLR® assay system). The luminescence ratios of Renilla-luciferase/Firefly-luciferase were calculated against the control of untreated aiRNAs.

Preparation of Transfection Mixture

The transfection mixtures A & B were prepared separately: 25 uL of two-component mixture-A contained 0.2 uL Lipofectamine-2000 reagent and the remaining volume of OptiMEM media; 25 uL of three-component mixture-B contained 200 ng of reporter plasmid DNA (N or P), 2.5 uL single dose testing concentration of corresponding aiRNA-cholesterol conjugate in PBS buffer plus the remaining volume of OptiMEM media. Mix both A and B solutions in a 1:1 ratio, before adding 50 uL resultant transfection mixture into each well incubated for 5 min at room temperature.

Screening Results

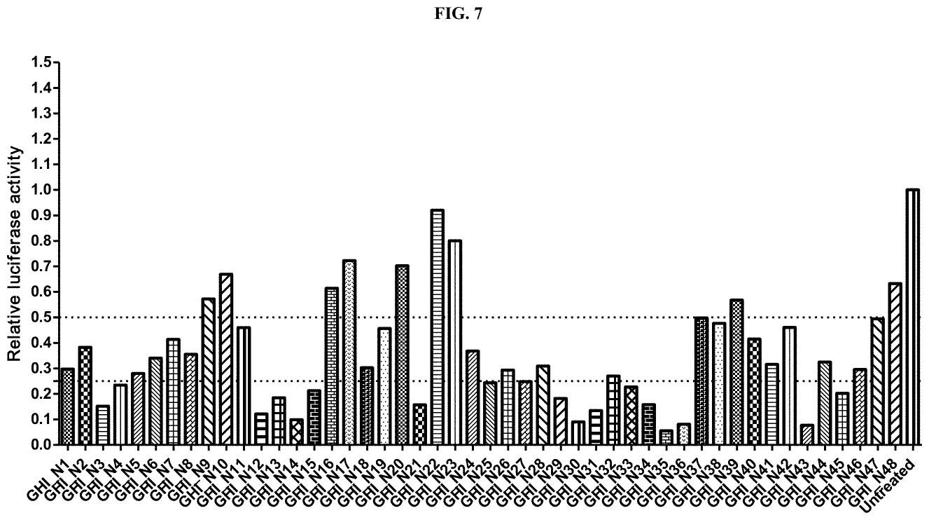

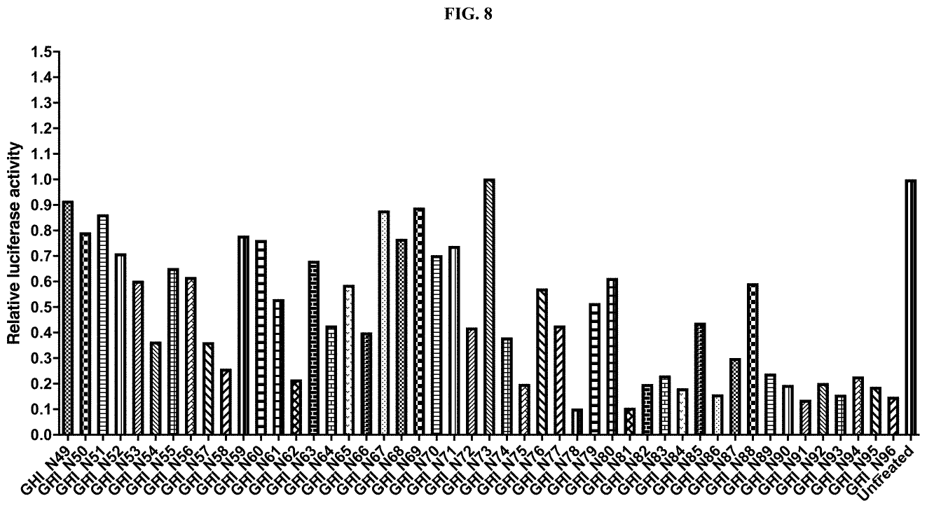

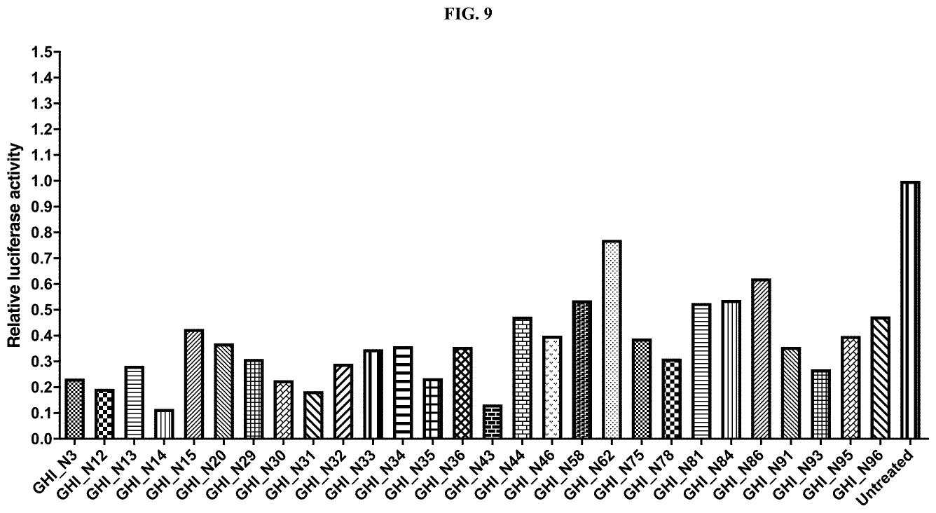

The results of all designed candidate N targeting aiRNAs (GHI_N1 to GHI_N96) at concentration of 1 nM are showed in FIG. 7 and FIG. 8. The ratios of Luc/Rluc were calculated against the mock-transfected control. Wherein the screened super N targeting aiRNAs are further screened at concentration at 100 pM. The results are showed in FIG. 9. Furthermore, the most effective ones were chosen for determination of IC₅₀ concentrations, and the results are showed in below Table 1.

TABLE 1
IC₅₀ concentrations of the most effective N targeting aiRNAs

| Duplex ID | IC₅₀ (pM) |
|---|---|
| GHI_N14 | 2.48 |
| GHI_N30 | 12.21 |
| GHI_N35 | 22.54 |
| GHI_N43 | 5.8 |

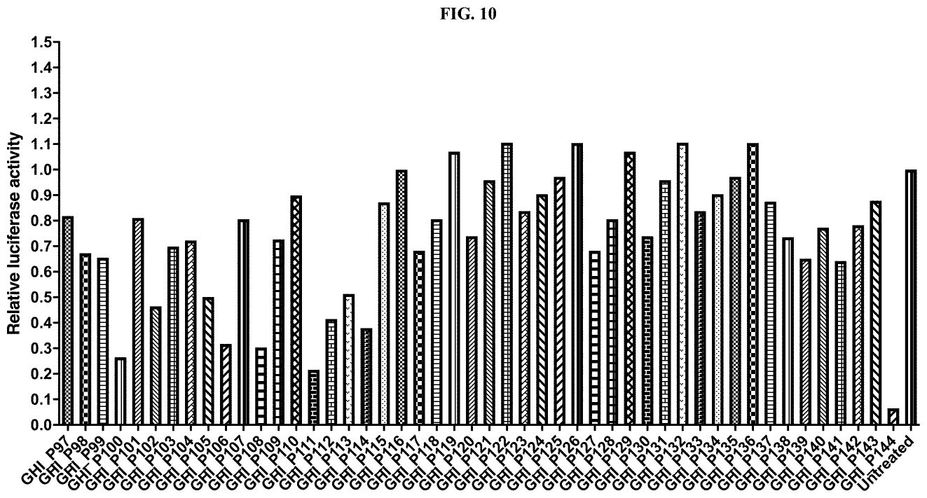

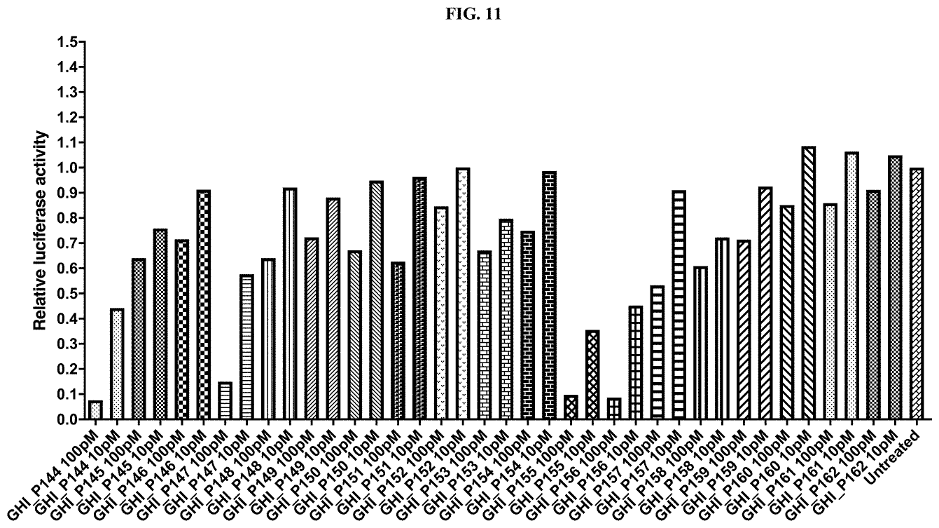

The results of designed candidate RdRp targeting aiRNAs (GHI_P97 to GHI_P144) at concentration of 1 nM are showed in FIG. 10, and the further screening results of designed candidate RdRp targeting aiRNAs (GHI_P144 to GHI_P162) at concentration of 100 pM and 10 pM are showed in FIG. 11. The ratios of Luc/Rluc were calculated against the mock-transfected control. Furthermore, the most effective ones were chosen for determination of IC₅₀ concentrations, and the results are showed in below Table 2.

TABLE 2
IC₅₀ concentrations of the most effective RdRp targeting aiRNAs

| Duplex ID | IC₅₀ (pM) |
|---|---|
| GHI_P144 | 45.48 |
| GHI_P147 | 51.34 |
| GHI_P155 | 17.08 |
| GHI_P156 | 35.2 |

### Example 3: In Vitro SARS-Cov-2 Viral Infection on Assay of N Targeting aiRNAs (GHI_N35, GHI_N43 and GHI_N81) and RdRp Targeting aiRNAs (GHI_P144, GHI_P147, GHI_P155 and GHI_P156) in Vero E6 Cells

Materials

Vero E6 cells were maintained in Eagle's Minimum Essential Medium (EMEM; Gibco Invitrogen) supplemented with 10% fetal bovine serum (FBS; Gibco Invitrogen) in a humidified atmosphere containing 5% CO₂ at 37° C.

The clinical isolate 2019-nCoV (SARS-CoV-2) was propagated in Vero E6 cells, and the virus titer was determined by measuring 50% tissue culture infectious dose (TCID₅₀) using immunofluorescence assay. All infection experiments were carried out in a Biosafety Level 3 (BLS-3) laboratory.

Each aiRNA compound or a combination of 1:1 ratio of aiRNA N43 and aiRNA P155 to be tested was dissolved in saline at a concentration of 1 mM as the stock solution. Five serial dilutions with concentrations at 100 μM, 33 μM, 10 μM, 3.3 μM, 1 μM, were prepared from the stock solution.

Evaluation Methods

Vero E6 (10,000 cells/well) cells cultured in EMEM (10% FBS) were inoculated into a 96-well plate and incubate in a humidified atmosphere containing 5% CO₂ at 37° C. for 24 hours. Samples of 10 μL of the above 1-100 μM dilutions were taken at each concentration point, and added into the 100 μL well plate to pretreat the cells for at least 48 hours; 1X Buffer was set as a negative control. The concentrations of test compounds after addition were as follows: 10 μM, 3.3 μM, 1 μM, 333 nM, 100 nM and 0.

Aliquots of SARS-CoV-2 virus (MOI of 0.05) were added to infect the treated cell populations for 2 hours. The virus-aiRNA compounds mixture was then removed, and the cells were further cultured with fresh medium containing the aiRNA compounds (concentration same with the pretreatment). After 48 hours of incubation, the cell supernatant was collected and lysed in lysis buffer (Takara, 9766) for quantitative analysis.

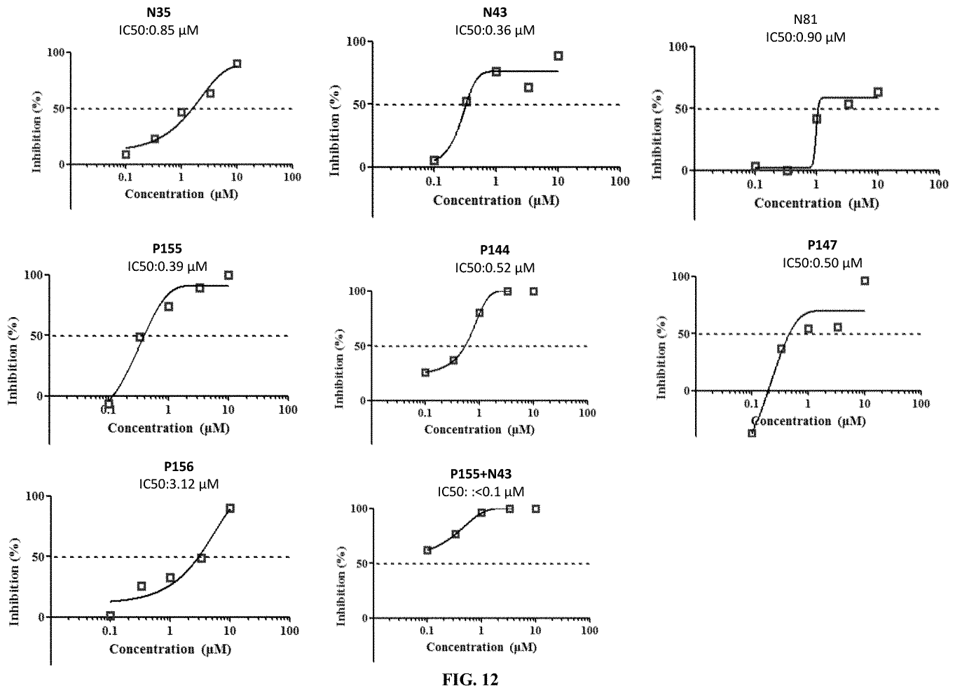

After samples of 100 μL of cell culture supernatant were collected from each cell population, viral RNA was isolated using MiniBEST Universal RNA Extraction Kit (TaKaRa, 9766). Viral RNA was eluted in 30 μL ribonuclease-free water. Reverse transcription was performed with a PrimeScript RT Reagent Kit with gDNA Eraser (Takara RR047A); qRT-PCR was performed on StepOnePlusTM Real-time PCR system (Applied Biosystem) with TB Green Premix Ex Taq II (Takara RR820A). The dose-response curves were plotted from viral RNA copies versus the drug concentrations using GraphPad Prism®. Results are shown in FIG. 12.

### Example 4: Inhibition of SARS-COV-2 Viral Infection and Replication In Vitro by aiRNAs

Designed test combination (GH543) was a combination of 1:1 ratio of aiRNA N43 compound and aiRNA P155 compound. The test evaluated the ability of the compound to inhibit virus production in Ver E6 cell culture. This assay was a two-step process, where the virus was first produced in cultures containing the antiviral substance at varying dilutions, followed later by titration of the samples for virus titer by endpoint dilution in 96-well microplates. Eight dilutions of the test compound were assayed, and the effective antiviral concentration determined by regression analysis. The toxicity of the test compound was determined in parallel.

Materials and Methods

SARS-CoV-2, USA-WA1/2020 stocks were prepared by passaging the virus in African green monkey kidney epithelial cell line (Vero 76) using test media of modified eagle's medium (MEM) supplemented with 2% heat-inactivated fetal bovine serum (FBS) and 50 g/mL gentamicin. Both aiRNA-1 (100 μM) and aiRNA-2 (100 μM) were mixed at a 1:1 ratio to prepare the 50 μM GH543. A 29.1 μM solution was then prepared in 1×RNAi buffer. Eight serial half-log dilutions were performed in 1x RNAi buffer, then 10 μL of each prepared concentration was added to 40 μL reduced serum MEM (OptiMEM). This solution was mixed with the prepared GH543 solutions at a 1:1 ratio (50 μL+50 μL). The compound was incubated for 20 minutes at room temperature. Each prepared dilution was then added in 13.75 μL for 5 wells of a 96-well plate containing 186.25 μL test media with 80-100% confluent Vero E6 cells. The final highest concentration on the plate was 200 nM.

The test combination (GH543) was prepared as above. Compound dilutions were incubated with cells for 24 hours prior to infection with SARS-CoV-2. SARS-CoV-2 was prepared to achieve the lowest possible multiplicity of infection (MOI of 0.001) that would yield >80% cytopathic effect (CPE) within 5 days. The protease inhibitor (M128533) was tested in parallel as a positive control, and in a similar way as test compound pre-treatment assay, i.e., 8 half log serial dilutions from start dose as 100 ug/mL. Plates were incubated at 37° C. and at 5% CO₂.

On day 3 post-infection supernatant fluid from each compound concentration was collected and tested for virus titer using a standard endpoint dilution CCID₅₀ assay and titer calculations using the Reed-Muench (1948) equation (Reed, L. J., 1938).

The in vitro SARS-COV-2 viral titer yield reductions by GH543 and positive control M128533 were summarized in Table 3. The concentration of compound required to reduce virus yield by 1 log 10 (EC₉₀) was calculated by regression analysis, and concentration of the test compound that would cause 50% cell death in the absence of virus (CC₅₀) was calculated in Table 4. The selective index of SI₉₀ was calculated as CC₅₀ divided by EC₉₀.

TABLE 3
In vitro SARS COV-2 titer yield reduction results by VYR assay

| GH543^{a} (nM) | Virus Titer |
|---|---|
| 200 | 6.7 |
| 63 | 5.3 |
| 20 | 3.3 |
| 6.3 | 1 |
| 2 | <0.7 |
| 0.6 | <0.7 |
| 0.2 | 5.7 |
| 0.06 | 6.7 |

^{a}Virus titer for GH543 VYR as CCID₅₀ per 0.1 mL, average virus control titers were 6.3 for the pre-treatment assay and 4.7 for the post-treatment assay.
^{b}Virus titer for M128533 VYR as CCID₅₀ per 0.1 mL, average virus control titer was 6.0.

TABLE 4
In vitro anti SARS COV-2 activity by VYR assay

| Compound | EC₉₀ | CC₅₀ | SI₉₀ |
|---|---|---|---|
| GH543 | 0.22 | >200 | >920 |
| M128533 | 14 | 91 | >6.4 |

The test compound GH543 was nontoxic (CC₅₀, >200 nM) and active against SARS-CoV-2, and showed significant virus titer reductions (EC₉₀, 0.22 nM), though the dose-response was not typical and higher concentrations did not inhibit CPE fully as lower concentrations did (Table 4, ignored for EC₉₀ calculation). The selective index ratio was high (SI₉₀>920) which is a good indication for a more effective and safer drug would be during in vivo treatment for a given viral infection. The positive control compound M128533 performed as expected.

GH543 reduces significantly virus infection and replication (EC₉₀=0.22 nM) compare to positive control (EC₉₀=14 nM), and the test compound is non-toxic to cells. Study provided evidence that could be a tool for inhibition of SARS-CoV-2.

### REFERENCES CITED

Baranov, P. V., Henderson, C. M., Anderson, C. B., Gesteland, R. F., Atkins, J. F., Howard, M. T., (2005). Programmed ribosomal frameshifting in decoding the SARS-CoV genome. Virology, 332, 498-510.
Beaucage, S. L., Caruthers, M. H. (1981). Deoxynucleoside phosphoramidites—A new class of key intermediates for deoxypolynucleotide synthesis. Tetrahedron Lett. 22, 1859-1862.
Behlke, M. A. (2008). Chemical Modification of siRNAs for In Vivo Use. Oligonucleotides 18, 305-320.
Chan, J. F. et al. (2015). Middle East respiratory syndrome coronavirus: another zoonotic betacoronavirus causing SARS-like disease. Clin. Microbiol. Rev., 28, 465-522.
Chang, C. I., Yoo, J. W., Hong, S. W., Lee, S. E., Kang, H. S., Sun, X., Rogoff, H. A., Ban, C., Kim, S., Li, C. J., Lee, D. K. (2009). Asymmetric shorter-duplex siRNA structures trigger efficient gene silencing with reduced nonspecific effects. Mol. Ther. 17, 725-732.
Davidson, B. L., McCray, P. B. Jr. (2011). Current prospects for RNA interference-based therapies. Nat. Rev. Genet. 12, 329-340.
Davis, M. E., Zuckerman, J. E., Choi, C. H. J., Seligson, D., Tolcher, A., Alabi, C. A., Yen, Y., Heidel, J. D., Ribas, A. (2010). Evidence of RNAi in humans from systemically administered siRNA via targeted nanoparticles. Nature 464, 1067-1070.
Elbashir, S. M., Lendeckel, W., Tuschl, T. (2001). RNA interference is mediated by 21- and 22-nucleotide RNAs. Genes Dev. 15, 188-200.
Fabian, M. R., Sonenberg, N., Filipowicz, W. (2010). Regulation of mRNA translation and stability by microRNAs. Annu. Rev. Biochem. 79, 351-379.
Fisher, D., Heymann, D. (2020). Q&A: The novel coronavirus outbreak causing COVID-19. BMC Med, 18, 57.
Jackson, A. L., Bartz, S. R., Schelter, J., Kobayashi, S. V., Burchard, J., Mao, M., Li, B., Cavet, G., Linsley, P. S., (2003). Expression profiling reveals off-target gene regulation by RNAi. Nat Biotechnol. 21, 635-637.
Jinek, M., Doudna, J. A. (2009). A three-dimensional view of the molecular machinery of RNA interference. Nature 457, 405-412.
Kleinman, M. E., Yamada, K., Takeda, A., Chandrasekaran, V., Nozaki, M., Baffi, J. Z., Albuquerque, R. J., Yamasaki, S., Itaya, M., Pan, Y., Appukuttan, B. (2008). Sequence- and target-independent angiogenesis suppression by siRNA via TLR3. Nature 452, 591-597.
Lu, M., Zhang, Q., Deng, M., Miao, J., Guo, Y., Gao, W., Cui, Q. (2008). An analysis of human microRNA and disease associations. PLoS One 3.10 e3420.
Marra Jones, S. J., Astell, et al. (2003). The genome sequence of the SARS-associated coronavirus. Science, 300, 1399-1404.
Masters, P. S., (2006). The molecular biology of coronaviruses. Adv. Virus Res., 66, 193-292.
Peiris, J. S., Guan, Y., Yuen, K. Y., (2004). Severe acute respiratory syndrome. Nat. Med., 10, S88-S97.
Rayner, S., Brignac, S., Bumeister, R., Belosludtsev, Y., Ward, T., Grant, O., O'Brien, K., Evans, G. A., Garner, H. R. (1998). Mermade: An Oligodeoxyribonucleotide Synthesizer for High Throughput Oligonucleotide Production in Dual 96-Well Plates. Genome Res. 8, 741-747.
Reese, C. B. (2005). Oligo- and poly-nucleotides: 50 years of chemical synthesis. Org. Biomol. Chem. 3, 3851-3868.
Scacheri, P. C., Rozenblatt-Rosen, O., Caplen, N.J., Wolfsberg, T. G., Umayam, L., Lee, J. C., Hughes, C. M., Shanmugam, K. S., Bhattacharjee, A., Meyerson, M., Collins, F. S. (2004). Short interfering RNAs can induce unexpected and divergent changes in the levels of untargeted proteins in mammalian cells. Proc. Natl. Acad. Sci. U.S.A 101, 1892-1897.
Shabalina, S. A., Koonin, E. V. (2008). Origins and evolution of eukaryotic RNA interference. Trends Ecol. Evol. 23, 578-587.
Sun, X., Rogoff, H. A., Li, C. J., (2008). Asymmetric RNA duplexes mediate RNA interference in mammalian cells. Nat. Biotechnol. 26, 1379-1382.
van Boheemen, S. et al. (2012). Genomic characterization of a newly discovered coronavirus associated with acute respiratory distress syndrome in humans. mBio 3, e00473-12.
Wilson R. C., Doudna, J. A. (2013). Molecular mechanisms of RNA interference. Annu Rev Biophys. 42, 217-239.
Xu, X., Liu, Y., Weiss, S., Arnold, E., Sarafianos, S. G., Ding, J., (2003). Molecular model of SARS coronavirus polymerase: implications for biochemical functions and drug design. Nucleic Acids Res., 31, 7117-7130.
Zhou P, Yang X, Wang X, et al. (2020). A pneumonia outbreak associated with a new coronavirus of probable bat origin. Nature, 579, 270-273.
Zhu, N., Zhang, D., Wang, W., Li, X., Yang, B., Song, J., Zhao, X., Huang, B., Shi, W., Lu, R., et al. (2020). A novel coronavirus from patients with pneumonia in China, 2019. N Engl J Med. 382, 727-733.
Sun, X., Rogoff, H. & Li, C. (2008). Asymmetric RNA duplexes mediate RNA interference in mammalian cells. Nat. Biotechnol., 26, 1379-1382.
Kunisaki, K. M., and Janoff, E. N. (2009). Influenza in immunosuppressed populations: a review of infection frequency, morbidity, mortality, and vaccine responses. Lancet Infect. Dis. 9, 493-504.
Hodgson, J. (2020). The pandemic pipeline. Nat. Biotechnol. 38, 523-532.
Lu, H. (2020). Drug treatment options for the 2019-new coronavirus (2019-nCoV). Biosci.
Levanova, A., and Poranen, M. M. (2018). RNA interference as a prospective tool for the control of human viral infections. Front. Microbiol. 9, 2151.
Lam, J. K., Chow, M. Y., Zhang, Y., and Leung, S. W. (2015). siRNA versus miRNA as therapeutics for gene silencing. Mol. Ther. Nucleic Acids 4, e252.
Borenfreund E., Puerner J. A. (1984) A simple quantitative procedure using monolayer cultures for cytotoxicity assays (HTDNR90). Journal of Tissue Culture Methods 9(1):7-9.
Borenfreund E., Puerner J. A. (1984) A simple quantitative procedure using monolayer cultures for cytotoxicity assays (HTDNR90). Journal of Tissue Culture Methods 9(1):7-9.

1. (canceled)

2. (canceled)

3. A gene silencing agent for treating COVID-19, wherein the gene silencing agent is a small interfering RNA duplex, comprising an antisense strand having at least 15 or more contiguous nucleotides selected from the group consisting of SEQ ID NOs: 211 to 420, and a sense strand substantially complementary to the antisense strand, the sense strand forming a double-stranded region with the antisense strand.

4. The agent of claim 3, wherein the antisense strand has a length of 15 to 30 nucleotides, the sense strand has a length of 9-29 nucleotides, both ends included.

5. The agent of claim 3, wherein the said antisense strand has a length of 23 nucleotides and the sense strand has a length of 12, 13, 14, 15, 16, 17, 18, 19, 20, 21, 22 or 23 nucleotides.

6. The agent of claim 3, wherein the said antisense strand has a length of 21 nucleotides and the sense strand has a length of 10, 11, 12, 13, 14, 15, 16, 17, 18, 19, 20 or 21 nucleotides.

7. The agent of claim 3, wherein the said antisense strand has a length of 19 nucleotides and the sense strand has a length of 10, 11, 12, 13, 14, 15, 16, 17, 18 or 19 nucleotides.

8. The agent of claim 3, wherein the antisense strand has overhang(s) at the 3′ and/or 5′ ends, wherein the overhang at the 3′ and/or 5′ ends has a length of 1, 2, 3, 4, 5 or 6 nucleotides.

9. The agent of claim 3, wherein the antisense strand has overhang at 3′ end and has a 5′blunt end, wherein the overhang at 3′ end has a length of 2, 3, 4, 5, 6, 7, 8, 9, 10, 11, 12 or 13 nucleotides.

10. A gene silencing agent for treating COVID-19, wherein said gene silencing agent is a small interfering RNA duplex, comprising an antisense strand comprising a nucleotide sequence that is essentially identical to a sequence selected from the group consisting of SEQ ID NOs: 211 to 420, and a sense strand comprising a nucleotide sequence that is essentially identical to a corresponding sequence selected from the group consisting of SEQ ID NOs: 421 to 630, the sense strand forming a double-stranded region with the antisense strand.

11. The gene silencing agent of claim 10, wherein said antisense strand comprises the sequence of 5′-AACGAUUGCAGCAUUGUUAGC-3′ (SEQ ID NO:253), and said sense strand comprises the sequence of 5′-AACAAUGCUGCAAUC-3′ (SEQ ID NO:463).

12. The gene silencing agent of claim 10, wherein said antisense strand comprises the sequence of 5′-AACUUUAUCAUUGUAGAUGUC-3′ (SEQ ID NO:365), and said sense strand comprises the sequence of 5′-AUCUACAAUGAUAAA-3′ (SEQ ID NO:575).

13. The gene silencing agent of claim 10, wherein said antisense strand comprises the sequence of 5′-AAGCAUUGUUAGCAGGAUUGC-3′ (SEQ ID NO:224), and said sense strand comprises the sequence of 5′-AUCCUGCUAACAAUG-3′ (SEQ ID NO:434).

14. The gene silencing agent of claim 10, wherein said antisense strand comprises the sequence of 5′-AACAAUUUGCGGCCAAUGUUU-3′ (SEQ ID NO:240), and said sense strand comprises the sequence of 5′-CAUUGGCCGCAAAUU-3′ (SEQ ID NO:450).

15. The gene silencing agent of claim 10, wherein said antisense strand comprises the sequence of 5′-AAUUGUUUGGAGAAAUCAUCC-3′ (SEQ ID NO:245), and said sense strand comprises the sequence of 5′-UGAUUUCUCCAAACA-3′ (SEQ ID NO:455).

16. The gene silencing agent of claim 10, wherein said antisense strand comprises the sequence of 5′-AAACCAGCUACUUUAUCAUUG-3′ (SEQ ID NO:366), and said sense strand comprises the sequence of 5′-UGAUAAAGUAGCUGG-3′ (SEQ ID NO:576).

17. The gene silencing agent of claim 10, wherein said antisense strand comprises the sequence of 5′-AAAUGUGUCACAAUUACCUUC-3′ (SEQ ID NO:354), and said sense strand comprises the sequence of 5′-GGUAAUUGUGACACA-3′ (SEQ ID NO:564).

18. The gene silencing agent of claim 10, wherein said antisense strand comprises the sequence of 5′-AACAGCUGGACAAUCCUUAAG-3′ (SEQ ID NO:357), and said sense strand comprises the sequence of 5′-AAGGAUUGUCCAGCU-3′ (SEQ ID NO:567).

19. The agent of claim 3, wherein at least one nucleotide of said antisense strand and/or sense strand is a modified nucleotide.

20. The agent of claim 3, wherein at least one of said antisense strand and sense strand is conjugated to a ligand comprising a lipid moiety such as a cholesterol moiety.

21. The agent of claim 3, wherein the said agent is capable of reducing the levels of SARS-CoV-2 protein, SARS-CoV-2 mRNA or SARS-CoV-2 titer in a cell in a subject.

22-35. (canceled)


SEQUENCE LISTING (630 sequences)

SEQ ID NO: 1 - RNA, 19 nt, SARS-CoV-2
  target sequence
aaagatcacattggcaccc

SEQ ID NO: 2 - RNA, 19 nt, SARS-CoV-2
  target sequence
taacacaagctttcggcag

SEQ ID NO: 3 - RNA, 19 nt, SARS-CoV-2
  target sequence
aaacagcaaactgtgactc

SEQ ID NO: 4 - RNA, 19 nt, SARS-CoV-2
  target sequence
aaggtttacccaataatac

SEQ ID NO: 5 - RNA, 19 nt, SARS-CoV-2
  target sequence
caccgctctcactcaacat

SEQ ID NO: 6 - RNA, 19 nt, SARS-CoV-2
  target sequence
tcaacatggcaaggaagac

SEQ ID NO: 7 - RNA, 19 nt, SARS-CoV-2
  target sequence
caaggaagaccttaaattc

SEQ ID NO: 8 - RNA, 19 nt, SARS-CoV-2
  target sequence
aaggcgttccaattaacac

SEQ ID NO: 9 - RNA, 19 nt, SARS-CoV-2
  target sequence
ttctactacctaggaactg

SEQ ID NO: 10 - RNA, 19 nt, SARS-CoV-2
  target sequence
cctatggtgctaacaaaga

SEQ ID NO: 11 - RNA, 19 nt, SARS-CoV-2
  target sequence
ctgagggagccttgaatac

SEQ ID NO: 12 - RNA, 19 nt, SARS-CoV-2
  target sequence
acattggcacccgcaatcc

SEQ ID NO: 13 - RNA, 19 nt, SARS-CoV-2
  target sequence
cccgcaatcctgctaacaa

SEQ ID NO: 14 - RNA, 19 nt, SARS-CoV-2
  target sequence
gcaatcctgctaacaatgc

SEQ ID NO: 15 - RNA, 19 nt, SARS-CoV-2
  target sequence
caacttcctcaaggaacaa

SEQ ID NO: 16 - RNA, 19 nt, SARS-CoV-2
  target sequence
aggcttctacgcagaaggg

SEQ ID NO: 17 - RNA, 19 nt, SARS-CoV-2
  target sequence
cagaggcggcagtcaagcc

SEQ ID NO: 18 - RNA, 19 nt, SARS-CoV-2
  target sequence
gcaacagttcaagaaattc

SEQ ID NO: 19 - RNA, 19 nt, SARS-CoV-2
  target sequence
ctagaatggctggcaatgg

SEQ ID NO: 20 - RNA, 19 nt, SARS-CoV-2
  target sequence
tggtaaaggccaacaacaa

SEQ ID NO: 21 - RNA, 19 nt, SARS-CoV-2
  target sequence
aaactgtcactaagaaatc

SEQ ID NO: 22 - RNA, 19 nt, SARS-CoV-2
  target sequence
ctgctgaggcttctaagaa

SEQ ID NO: 23 - RNA, 19 nt, SARS-CoV-2
  target sequence
ctgaggcttctaagaagcc

SEQ ID NO: 24 - RNA, 19 nt, SARS-CoV-2
  target sequence
aacgtactgccactaaagc

SEQ ID NO: 25 - RNA, 19 nt, SARS-CoV-2
  target sequence
acgtactgccactaaagca

SEQ ID NO: 26 - RNA, 19 nt, SARS-CoV-2
  target sequence
ccactaaagcatacaatgt

SEQ ID NO: 27 - RNA, 19 nt, SARS-CoV-2
  target sequence
gaacaaacccaaggaaatt

SEQ ID NO: 28 - RNA, 19 nt, SARS-CoV-2
  target sequence
ggaactaatcagacaagga

SEQ ID NO: 29 - RNA, 19 nt, SARS-CoV-2
  target sequence
ctaatcagacaaggaactg

SEQ ID NO: 30 - RNA, 19 nt, SARS-CoV-2
  target sequence
aaacattggccgcaaattg

SEQ ID NO: 31 - RNA, 19 nt, SARS-CoV-2
  target sequence
gcgcattggcatggaagtc

SEQ ID NO: 32 - RNA, 19 nt, SARS-CoV-2
  target sequence
acacaggtgccatcaaatt

SEQ ID NO: 33 - RNA, 19 nt, SARS-CoV-2
  target sequence
cacaggtgccatcaaattg

SEQ ID NO: 34 - RNA, 19 nt, SARS-CoV-2
  target sequence
cagaagaaacagcaaactg

SEQ ID NO: 35 - RNA, 19 nt, SARS-CoV-2
  target sequence
ggatgatttctccaaacaa

SEQ ID NO: 36 - RNA, 19 nt, SARS-CoV-2
  target sequence
tgatttctccaaacaattg

SEQ ID NO: 37 - RNA, 19 nt, SARS-CoV-2
  target sequence
ccgcattacgtttggtgga

SEQ ID NO: 38 - RNA, 19 nt, SARS-CoV-2
  target sequence
agtaaccagaatggagaac

SEQ ID NO: 39 - RNA, 19 nt, SARS-CoV-2
  target sequence
cccaataatactgcgtctt

SEQ ID NO: 40 - RNA, 19 nt, SARS-CoV-2
  target sequence
aaggaagaccttaaattcc

SEQ ID NO: 41 - RNA, 19 nt, SARS-CoV-2
  target sequence
gatgaccaaattggctact

SEQ ID NO: 42 - RNA, 19 nt, SARS-CoV-2
  target sequence
gtccaagatggtatttcta

SEQ ID NO: 43 - RNA, 19 nt, SARS-CoV-2
  target sequence
gctaacaatgctgcaatcg

SEQ ID NO: 44 - RNA, 19 nt, SARS-CoV-2
  target sequence
aaggaacaacattgccaaa

SEQ ID NO: 45 - RNA, 19 nt, SARS-CoV-2
  target sequence
gttcaagaaattcaactcc

SEQ ID NO: 46 - RNA, 19 nt, SARS-CoV-2
  target sequence
ttgaaccagcttgagagca

SEQ ID NO: 47 - RNA, 19 nt, SARS-CoV-2
  target sequence
actaagaaatctgctgctg

SEQ ID NO: 48 - RNA, 19 nt, SARS-CoV-2
  target sequence
tctaagaagcctcggcaaa

SEQ ID NO: 49 - RNA, 19 nt, SARS-CoV-2
  target sequence
ggcatcatatgggttgcaa

SEQ ID NO: 50 - RNA, 19 nt, SARS-CoV-2
  target sequence
gcatcatatgggttgcaac

SEQ ID NO: 51 - RNA, 19 nt, SARS-CoV-2
  target sequence
catcatatgggttgcaact

SEQ ID NO: 52 - RNA, 19 nt, SARS-CoV-2
  target sequence
aagcctcttctcgttcctc

SEQ ID NO: 53 - RNA, 19 nt, SARS-CoV-2
  target sequence
caagcctcttctcgttcct

SEQ ID NO: 54 - RNA, 19 nt, SARS-CoV-2
  target sequence
acccgcaatcctgctaaca

SEQ ID NO: 55 - RNA, 19 nt, SARS-CoV-2
  target sequence
ttacccaataatactgcgt

SEQ ID NO: 56 - RNA, 19 nt, SARS-CoV-2
  target sequence
agaccttaaattccctcga

SEQ ID NO: 57 - RNA, 19 nt, SARS-CoV-2
  target sequence
ggcgttccaattaacacca

SEQ ID NO: 58 - RNA, 19 nt, SARS-CoV-2
  target sequence
atcgtgctacaacttcctc

SEQ ID NO: 59 - RNA, 19 nt, SARS-CoV-2
  target sequence
tgagggagccttgaataca

SEQ ID NO: 60 - RNA, 19 nt, SARS-CoV-2
  target sequence
catcatatgggttgcaact

SEQ ID NO: 61 - RNA, 19 nt, SARS-CoV-2
  target sequence
caatagcagtccagatgac

SEQ ID NO: 62 - RNA, 19 nt, SARS-CoV-2
  target sequence
agcagtccagatgaccaaa

SEQ ID NO: 63 - RNA, 19 nt, SARS-CoV-2
  target sequence
aaagatctcagtccaagat

SEQ ID NO: 64 - RNA, 19 nt, SARS-CoV-2
  target sequence
ctgcgtcttggttcaccgc

SEQ ID NO: 65 - RNA, 19 nt, SARS-CoV-2
  target sequence
aattggctactaccgaaga

SEQ ID NO: 66 - RNA, 19 nt, SARS-CoV-2
  target sequence
gaagagctaccagacgaat

SEQ ID NO: 67 - RNA, 19 nt, SARS-CoV-2
  target sequence
caaagacggcatcatatgg

SEQ ID NO: 68 - RNA, 19 nt, SARS-CoV-2
  target sequence
agacggcatcatatgggtt

SEQ ID NO: 69 - RNA, 19 nt, SARS-CoV-2
  target sequence
agacgaattcgtggtggtg

SEQ ID NO: 70 - RNA, 19 nt, SARS-CoV-2
  target sequence
cgaattcgtggtggtgacg

SEQ ID NO: 71 - RNA, 19 nt, SARS-CoV-2
  target sequence
tcagtccaagatggtattt

SEQ ID NO: 72 - RNA, 19 nt, SARS-CoV-2
  target sequence
tcatcacgtagtcgcaaca

SEQ ID NO: 73 - RNA, 19 nt, SARS-CoV-2
  target sequence
ggacttccctatggtgcta

SEQ ID NO: 74 - RNA, 19 nt, SARS-CoV-2
  target sequence
ctggacttccctatggtgc

SEQ ID NO: 75 - RNA, 19 nt, SARS-CoV-2
  target sequence
ttcagcgttcttcggaatg

SEQ ID NO: 76 - RNA, 19 nt, SARS-CoV-2
  target sequence
gtcttggttcaccgctctc

SEQ ID NO: 77 - RNA, 19 nt, SARS-CoV-2
  target sequence
cttgacagattgaaccagc

SEQ ID NO: 78 - RNA, 19 nt, SARS-CoV-2
  target sequence
gcgcttcagcgttcttcgg

SEQ ID NO: 79 - RNA, 19 nt, SARS-CoV-2
  target sequence
ctcgttcctcatcacgtag

SEQ ID NO: 80 - RNA, 19 nt, SARS-CoV-2
  target sequence
gtaacacaagctttcggca

SEQ ID NO: 81 - RNA, 19 nt, SARS-CoV-2
  target sequence
aataagcatattgacgcat

SEQ ID NO: 82 - RNA, 19 nt, SARS-CoV-2
  target sequence
ttacaaacattggccgcaa

SEQ ID NO: 83 - RNA, 19 nt, SARS-CoV-2
  target sequence
cattggccgcaaattgcac

SEQ ID NO: 84 - RNA, 19 nt, SARS-CoV-2
  target sequence
gattacaaacattggccgc

SEQ ID NO: 85 - RNA, 19 nt, SARS-CoV-2
  target sequence
ttcggcagacgtggtccag

SEQ ID NO: 86 - RNA, 19 nt, SARS-CoV-2
  target sequence
gttcttcggaatgtcgcgc

SEQ ID NO: 87 - RNA, 19 nt, SARS-CoV-2
  target sequence
ccagcgcttcagcgttctt

SEQ ID NO: 88 - RNA, 19 nt, SARS-CoV-2
  target sequence
gcggtgatgctgctcttgc

SEQ ID NO: 89 - RNA, 19 nt, SARS-CoV-2
  target sequence
ctgaataagcatattgacg

SEQ ID NO: 90 - RNA, 19 nt, SARS-CoV-2
  target sequence
aagtcacaccttcgggaac

SEQ ID NO: 91 - RNA, 19 nt, SARS-CoV-2
  target sequence
agtcacaccttcgggaacg

SEQ ID NO: 92 - RNA, 19 nt, SARS-CoV-2
  target sequence
aacgtggttgacctacaca

SEQ ID NO: 93 - RNA, 19 nt, SARS-CoV-2
  target sequence
cgtggttgacctacacagg

SEQ ID NO: 94 - RNA, 19 nt, SARS-CoV-2
  target sequence
aaactcaagccttaccgca

SEQ ID NO: 95 - RNA, 19 nt, SARS-CoV-2
  target sequence
tcacaccttcgggaacgtg

SEQ ID NO: 96 - RNA, 19 nt, SARS-CoV-2
  target sequence
gttgacctacacaggtgcc

SEQ ID NO: 97 - RNA, 19 nt, SARS-CoV-2
  target sequence
cttacatagctctagactt

SEQ ID NO: 98 - RNA, 19 nt, SARS-CoV-2
  target sequence
ctgtcaaacccggtaattt

SEQ ID NO: 99 - RNA, 19 nt, SARS-CoV-2
  target sequence
tttcaaactgtcaaacccg

SEQ ID NO: 100 - RNA, 19 nt, SARS-CoV-2
  target sequence
ggtgacatggtaccacata

SEQ ID NO: 101 - RNA, 19 nt, SARS-CoV-2
  target sequence
acatggtaccacatatatc

SEQ ID NO: 102 - RNA, 19 nt, SARS-CoV-2
  target sequence
gacatggtaccacatatat

SEQ ID NO: 103 - RNA, 19 nt, SARS-CoV-2
  target sequence
tgcattctgcattgtgcaa

SEQ ID NO: 104 - RNA, 19 nt, SARS-CoV-2
  target sequence
attctgcattgtgcaaact

SEQ ID NO: 105 - RNA, 19 nt, SARS-CoV-2
  target sequence
cgtcaacgtcttactaaat

SEQ ID NO: 106 - RNA, 19 nt, SARS-CoV-2
  target sequence
atatatcacgtcaacgtct

SEQ ID NO: 107 - RNA, 19 nt, SARS-CoV-2
  target sequence
taagtttagaatagacggt

SEQ ID NO: 108 - RNA, 19 nt, SARS-CoV-2
  target sequence
tcacgtcaacgtcttacta

SEQ ID NO: 109 - RNA, 19 nt, SARS-CoV-2
  target sequence
ttagaatagacggtgacat

SEQ ID NO: 110 - RNA, 19 nt, SARS-CoV-2
  target sequence
atacgccaacttaggtgaa

SEQ ID NO: 111 - RNA, 19 nt, SARS-CoV-2
  target sequence
agacggtgacatggtacca

SEQ ID NO: 112 - RNA, 19 nt, SARS-CoV-2
  target sequence
tttgatgaaggtaattgtg

SEQ ID NO: 113 - RNA, 19 nt, SARS-CoV-2
  target sequence
gaatagacggtgacatggt

SEQ ID NO: 114 - RNA, 19 nt, SARS-CoV-2
  target sequence
agaatagacggtgacatgg

SEQ ID NO: 115 - RNA, 19 nt, SARS-CoV-2
  target sequence
caacgtcttactaaataca

SEQ ID NO: 116 - RNA, 19 nt, SARS-CoV-2
  target sequence
acatacaattgttgtgatg

SEQ ID NO: 117 - RNA, 19 nt, SARS-CoV-2
  target sequence
tacaaaccacgccaggtag

SEQ ID NO: 118 - RNA, 19 nt, SARS-CoV-2
  target sequence
tcacatacaattgttgtga

SEQ ID NO: 119 - RNA, 19 nt, SARS-CoV-2
  target sequence
acaaaccacgccaggtagt

SEQ ID NO: 120 - RNA, 19 nt, SARS-CoV-2
  target sequence
tacttgtcacatacaattg

SEQ ID NO: 121 - RNA, 19 nt, SARS-CoV-2
  target sequence
tgtgatgatgattatttca

SEQ ID NO: 122 - RNA, 19 nt, SARS-CoV-2
  target sequence
ttaggtgaacgtgtacgcc

SEQ ID NO: 123 - RNA, 19 nt, SARS-CoV-2
  target sequence
aaagaaatacttgtcacat

SEQ ID NO: 124 - RNA, 19 nt, SARS-CoV-2
  target sequence
ctatattaaccttgaccag

SEQ ID NO: 125 - RNA, 19 nt, SARS-CoV-2
  target sequence
acataccacccaaattgtg

SEQ ID NO: 126 - RNA, 19 nt, SARS-CoV-2
  target sequence
cacccaaattgtgttaact

SEQ ID NO: 127 - RNA, 19 nt, SARS-CoV-2
  target sequence
tcatacaaaccacgccagg

SEQ ID NO: 128 - RNA, 19 nt, SARS-CoV-2
  target sequence
cagacataccacccaaatt

SEQ ID NO: 129 - RNA, 19 nt, SARS-CoV-2
  target sequence
ctgtgatgccatgcgaaat

SEQ ID NO: 130 - RNA, 19 nt, SARS-CoV-2
  target sequence
ttgtgcaaactttaatgtt

SEQ ID NO: 131 - RNA, 19 nt, SARS-CoV-2
  target sequence
aaacagtacaattctgtga

SEQ ID NO: 132 - RNA, 19 nt, SARS-CoV-2
  target sequence
atatattacgcgtatacgc

SEQ ID NO: 133 - RNA, 19 nt, SARS-CoV-2
  target sequence
acccaaattgtgttaactg

SEQ ID NO: 134 - RNA, 19 nt, SARS-CoV-2
  target sequence
tgcgaaatgctggtattgt

SEQ ID NO: 135 - RNA, 19 nt, SARS-CoV-2
  target sequence
ttacattaagtgggatttg

SEQ ID NO: 136 - RNA, 19 nt, SARS-CoV-2
  target sequence
tgacattagataatcaaga

SEQ ID NO: 137 - RNA, 19 nt, SARS-CoV-2
  target sequence
cggtgatttcatacaaacc

SEQ ID NO: 138 - RNA, 19 nt, SARS-CoV-2
  target sequence
ctgcacttactaacaatgt

SEQ ID NO: 139 - RNA, 19 nt, SARS-CoV-2
  target sequence
gtgtactgacattagataa

SEQ ID NO: 140 - RNA, 19 nt, SARS-CoV-2
  target sequence
acattagataatcaagatc

SEQ ID NO: 141 - RNA, 19 nt, SARS-CoV-2
  target sequence
tcattgttaatgcctatat

SEQ ID NO: 142 - RNA, 19 nt, SARS-CoV-2
  target sequence
cataatcaggatgtaaact

SEQ ID NO: 143 - RNA, 19 nt, SARS-CoV-2
  target sequence
cacatgttgacactgactt

SEQ ID NO: 144 - RNA, 19 nt, SARS-CoV-2
  target sequence
gaaggtaattgtgacacat

SEQ ID NO: 145 - RNA, 19 nt, SARS-CoV-2
  target sequence
taacaaagacttctatgac

SEQ ID NO: 146 - RNA, 19 nt, SARS-CoV-2
  target sequence
tgttacgatggtggctgta

SEQ ID NO: 147 - RNA, 19 nt, SARS-CoV-2
  target sequence
cttaaggattgtccagctg

SEQ ID NO: 148 - RNA, 19 nt, SARS-CoV-2
  target sequence
gccttacattaagtgggat

SEQ ID NO: 149 - RNA, 19 nt, SARS-CoV-2
  target sequence
cccaaattgtgttaactgt

SEQ ID NO: 150 - RNA, 19 nt, SARS-CoV-2
  target sequence
gataaacgcactacgtgct

SEQ ID NO: 151 - RNA, 19 nt, SARS-CoV-2
  target sequence
cacttactaacaatgttgc

SEQ ID NO: 152 - RNA, 19 nt, SARS-CoV-2
  target sequence
gttaatgcctatattaacc

SEQ ID NO: 153 - RNA, 19 nt, SARS-CoV-2
  target sequence
gagttcctgttgtagattc

SEQ ID NO: 154 - RNA, 19 nt, SARS-CoV-2
  target sequence
gttgtacataatcaggatg

SEQ ID NO: 155 - RNA, 19 nt, SARS-CoV-2
  target sequence
gacatctacaatgataaag

SEQ ID NO: 156 - RNA, 19 nt, SARS-CoV-2
  target sequence
caatgataaagtagctggt

SEQ ID NO: 157 - RNA, 19 nt, SARS-CoV-2
  target sequence
cgtctatgctttaaggcat

SEQ ID NO: 158 - RNA, 19 nt, SARS-CoV-2
  target sequence
caattgttgtgatgatgat

SEQ ID NO: 159 - RNA, 19 nt, SARS-CoV-2
  target sequence
gacttaacaaagccttaca

SEQ ID NO: 160 - RNA, 19 nt, SARS-CoV-2
  target sequence
ctaactaccaacatgaaga

SEQ ID NO: 161 - RNA, 19 nt, SARS-CoV-2
  target sequence
cttcacggaagagaggtta

SEQ ID NO: 162 - RNA, 19 nt, SARS-CoV-2
  target sequence
gtaatgtcatccctactat

SEQ ID NO: 163 - RNA, 19 nt, SARS-CoV-2
  target sequence
ggacacaactattacagtt

SEQ ID NO: 164 - RNA, 19 nt, SARS-CoV-2
  target sequence
acacaagaccatgttgaca

SEQ ID NO: 165 - RNA, 19 nt, SARS-CoV-2
  target sequence
caagaccatgttgacatac

SEQ ID NO: 166 - RNA, 19 nt, SARS-CoV-2
  target sequence
gaagatgaatttacacctt

SEQ ID NO: 167 - RNA, 19 nt, SARS-CoV-2
  target sequence
ttagacaatgctcaggtgt

SEQ ID NO: 168 - RNA, 19 nt, SARS-CoV-2
  target sequence
gatctgcacctctgaagac

SEQ ID NO: 169 - RNA, 19 nt, SARS-CoV-2
  target sequence
tggtacaggctggtaatgt

SEQ ID NO: 170 - RNA, 19 nt, SARS-CoV-2
  target sequence
aggttgatacagccaatcc

SEQ ID NO: 171 - RNA, 19 nt, SARS-CoV-2
  target sequence
atcctaagacacctaagta

SEQ ID NO: 172 - RNA, 19 nt, SARS-CoV-2
  target sequence
gtttgttcgcattcaacca

SEQ ID NO: 173 - RNA, 19 nt, SARS-CoV-2
  target sequence
ccaatttcactattaaggg

SEQ ID NO: 174 - RNA, 19 nt, SARS-CoV-2
  target sequence
tggcacagacttagaaggt

SEQ ID NO: 175 - RNA, 19 nt, SARS-CoV-2
  target sequence
caactattacagttaatgt

SEQ ID NO: 176 - RNA, 19 nt, SARS-CoV-2
  target sequence
ttaccacaactcttaatga

SEQ ID NO: 177 - RNA, 19 nt, SARS-CoV-2
  target sequence
accttgtggctatgaagta

SEQ ID NO: 178 - RNA, 19 nt, SARS-CoV-2
  target sequence
aattatgaacctctaacac

SEQ ID NO: 179 - RNA, 19 nt, SARS-CoV-2
  target sequence
tctaatcataatttcttgg

SEQ ID NO: 180 - RNA, 19 nt, SARS-CoV-2
  target sequence
aagtataagtttgttcgca

SEQ ID NO: 181 - RNA, 19 nt, SARS-CoV-2
  target sequence
tgttacaatggttcaccat

SEQ ID NO: 182 - RNA, 19 nt, SARS-CoV-2
  target sequence
gtttaccaatgtgctatga

SEQ ID NO: 183 - RNA, 19 nt, SARS-CoV-2
  target sequence
gattatgactgtgtctctt

SEQ ID NO: 184 - RNA, 19 nt, SARS-CoV-2
  target sequence
taccacaactcttaatgac

SEQ ID NO: 185 - RNA, 19 nt, SARS-CoV-2
  target sequence
cttaatgactttaaccttg

SEQ ID NO: 186 - RNA, 19 nt, SARS-CoV-2
  target sequence
atgaagtacaattatgaac

SEQ ID NO: 187 - RNA, 19 nt, SARS-CoV-2
  target sequence
aagtacaattatgaacctc

SEQ ID NO: 188 - RNA, 19 nt, SARS-CoV-2
  target sequence
acgtaccatattgggtagt

SEQ ID NO: 189 - RNA, 19 nt, SARS-CoV-2
  target sequence
tgtgatctgcacctctgaa

SEQ ID NO: 190 - RNA, 19 nt, SARS-CoV-2
  target sequence
gttcaccatctggtgttta

SEQ ID NO: 191 - RNA, 19 nt, SARS-CoV-2
  target sequence
ctacaagatgcttattaca

SEQ ID NO: 192 - RNA, 19 nt, SARS-CoV-2
  target sequence
taataagacagtaggtgag

SEQ ID NO: 193 - RNA, 19 nt, SARS-CoV-2
  target sequence
gcagacaacccttaagggt

SEQ ID NO: 194 - RNA, 19 nt, SARS-CoV-2
  target sequence
ctaaagaaactttgtattg

SEQ ID NO: 195 - RNA, 19 nt, SARS-CoV-2
  target sequence
gaatacaaaggtcctatta

SEQ ID NO: 196 - RNA, 19 nt, SARS-CoV-2
  target sequence
gtggaaatacccacaagtt

SEQ ID NO: 197 - RNA, 19 nt, SARS-CoV-2
  target sequence
ttaaatgggcagataacaa

SEQ ID NO: 198 - RNA, 19 nt, SARS-CoV-2
  target sequence
gttaacactccaacaaata

SEQ ID NO: 199 - RNA, 19 nt, SARS-CoV-2
  target sequence
ttaacactccaacaaatag

SEQ ID NO: 200 - RNA, 19 nt, SARS-CoV-2
  target sequence
ctggtgaagctgctaactt

SEQ ID NO: 201 - RNA, 19 nt, SARS-CoV-2
  target sequence
ggacaacagcagacaaccc

SEQ ID NO: 202 - RNA, 19 nt, SARS-CoV-2
  target sequence
agcagacaacccttaaggg

SEQ ID NO: 203 - RNA, 19 nt, SARS-CoV-2
  target sequence
gtgagtacactggtaatta

SEQ ID NO: 204 - RNA, 19 nt, SARS-CoV-2
  target sequence
gtgtggtcactataaacat

SEQ ID NO: 205 - RNA, 19 nt, SARS-CoV-2
  target sequence
ggtgctttacttacaaagt

SEQ ID NO: 206 - RNA, 19 nt, SARS-CoV-2
  target sequence
tacaaagtcctcagaatac

SEQ ID NO: 207 - RNA, 19 nt, SARS-CoV-2
  target sequence
gataacaactgttatcttg

SEQ ID NO: 208 - RNA, 19 nt, SARS-CoV-2
  target sequence
aacaaatagagttgaagtt

SEQ ID NO: 209 - RNA, 19 nt, SARS-CoV-2
  target sequence
tttaagaaaggtgttcaga

SEQ ID NO: 210 - RNA, 19 nt, SARS-CoV-2
  target sequence
cagtatgaacttaagcatg

SEQ ID NO: 211 - RNA, 21 nt, Artificial Sequence
  AS sequence
aagggugccaaugugaucuuu

SEQ ID NO: 212 - RNA, 21 nt, Artificial Sequence
  AS sequence
aacugccgaaagcuuguguua

SEQ ID NO: 213 - RNA, 21 nt, Artificial Sequence
  AS sequence
aagagucacaguuugcuguuu

SEQ ID NO: 214 - RNA, 21 nt, Artificial Sequence
  AS sequence
aaguauuauuggguaaaccuu

SEQ ID NO: 215 - RNA, 21 nt, Artificial Sequence
  AS sequence
aaauguugagugagagcggug

SEQ ID NO: 216 - RNA, 21 nt, Artificial Sequence
  AS sequence
aagucuuccuugccauguuga

SEQ ID NO: 217 - RNA, 21 nt, Artificial Sequence
  AS sequence
aagaauuuaaggucuuccuug

SEQ ID NO: 218 - RNA, 21 nt, Artificial Sequence
  AS sequence
aaguguuaauuggaacgccuu

SEQ ID NO: 219 - RNA, 21 nt, Artificial Sequence
  AS sequence
aacaguuccuagguaguagaa

SEQ ID NO: 220 - RNA, 21 nt, Artificial Sequence
  AS sequence
aaucuuuguuagcaccauagg

SEQ ID NO: 221 - RNA, 21 nt, Artificial Sequence
  AS sequence
aaguauucaaggcucccucag

SEQ ID NO: 222 - RNA, 21 nt, Artificial Sequence
  AS sequence
aaggauugcgggugccaaugu

SEQ ID NO: 223 - RNA, 21 nt, Artificial Sequence
  AS sequence
aauuguuagcaggauugcggg

SEQ ID NO: 224 - RNA, 21 nt, Artificial Sequence
  AS sequence
aagcauuguuagcaggauugc

SEQ ID NO: 225 - RNA, 21 nt, Artificial Sequence
  AS sequence
aauuguuccuugaggaaguug

SEQ ID NO: 226 - RNA, 21 nt, Artificial Sequence
  AS sequence
aacccuucugcguagaagccu

SEQ ID NO: 227 - RNA, 21 nt, Artificial Sequence
  AS sequence
aaggcuugacugccgccucug

SEQ ID NO: 228 - RNA, 21 nt, Artificial Sequence
  AS sequence
aagaauuucuugaacuguugc

SEQ ID NO: 229 - RNA, 21 nt, Artificial Sequence
  AS sequence
aaccauugccagccauucuag

SEQ ID NO: 230 - RNA, 21 nt, Artificial Sequence
  AS sequence
aauuguuguuggccuuuacca

SEQ ID NO: 231 - RNA, 21 nt, Artificial Sequence
  AS sequence
aagauuucuuagugacaguuu

SEQ ID NO: 232 - RNA, 21 nt, Artificial Sequence
  AS sequence
aauucuuagaagccucagcag

SEQ ID NO: 233 - RNA, 21 nt, Artificial Sequence
  AS sequence
aaggcuucuuagaagccucag

SEQ ID NO: 234 - RNA, 21 nt, Artificial Sequence
  AS sequence
aagcuuuaguggcaguacguu

SEQ ID NO: 235 - RNA, 21 nt, Artificial Sequence
  AS sequence
aaugcuuuaguggcaguacgu

SEQ ID NO: 236 - RNA, 21 nt, Artificial Sequence
  AS sequence
aaacauuguaugcuuuagugg

SEQ ID NO: 237 - RNA, 21 nt, Artificial Sequence
  AS sequence
aaaauuuccuuggguuuguuc

SEQ ID NO: 238 - RNA, 21 nt, Artificial Sequence
  AS sequence
aauccuugucugauuaguucc

SEQ ID NO: 239 - RNA, 21 nt, Artificial Sequence
  AS sequence
aacaguuccuugucugauuag

SEQ ID NO: 240 - RNA, 21 nt, Artificial Sequence
  AS sequence
aacaauuugcggccaauguuu

SEQ ID NO: 241 - RNA, 21 nt, Artificial Sequence
  AS sequence
aagacuuccaugccaaugcgc

SEQ ID NO: 242 - RNA, 21 nt, Artificial Sequence
  AS sequence
aaaauuugauggcaccugugu

SEQ ID NO: 243 - RNA, 21 nt, Artificial Sequence
  AS sequence
aacaauuugauggcaccugug

SEQ ID NO: 244 - RNA, 21 nt, Artificial Sequence
  AS sequence
aacaguuugcuguuucuucug

SEQ ID NO: 245 - RNA, 21 nt, Artificial Sequence
  AS sequence
aauuguuuggagaaaucaucc

SEQ ID NO: 246 - RNA, 21 nt, Artificial Sequence
  AS sequence
aacaauuguuuggagaaauca

SEQ ID NO: 247 - RNA, 21 nt, Artificial Sequence
  AS sequence
aauccaccaaacguaaugcgg

SEQ ID NO: 248 - RNA, 21 nt, Artificial Sequence
  AS sequence
aaguucuccauucugguuacu

SEQ ID NO: 249 - RNA, 21 nt, Artificial Sequence
  AS sequence
aaaagacgcaguauuauuggg

SEQ ID NO: 250 - RNA, 21 nt, Artificial Sequence
  AS sequence
aaggaauuuaaggucuuccuu

SEQ ID NO: 251 - RNA, 21 nt, Artificial Sequence
  AS sequence
aaaguagccaauuuggucauc

SEQ ID NO: 252 - RNA, 21 nt, Artificial Sequence
  AS sequence
aauagaaauaccaucuuggac

SEQ ID NO: 253 - RNA, 21 nt, Artificial Sequence
  AS sequence
aacgauugcagcauuguuagc

SEQ ID NO: 254 - RNA, 21 nt, Artificial Sequence
  AS sequence
aauuuggcaauguuguuccuu

SEQ ID NO: 255 - RNA, 21 nt, Artificial Sequence
  AS sequence
aaggaguugaauuucuugaac

SEQ ID NO: 256 - RNA, 21 nt, Artificial Sequence
  AS sequence
aaugcucucaagcugguucaa

SEQ ID NO: 257 - RNA, 21 nt, Artificial Sequence
  AS sequence
aacagcagcagauuucuuagu

SEQ ID NO: 258 - RNA, 21 nt, Artificial Sequence
  AS sequence
aauuugccgaggcuucuuaga

SEQ ID NO: 259 - RNA, 21 nt, Artificial Sequence
  AS sequence
aauugcaacccauaugaugcc

SEQ ID NO: 260 - RNA, 21 nt, Artificial Sequence
  AS sequence
aaguugcaacccauaugaugc

SEQ ID NO: 261 - RNA, 21 nt, Artificial Sequence
  AS sequence
aaaguugcaacccauaugaug

SEQ ID NO: 262 - RNA, 21 nt, Artificial Sequence
  AS sequence
aagaggaacgagaagaggcuu

SEQ ID NO: 263 - RNA, 21 nt, Artificial Sequence
  AS sequence
aaaggaacgagaagaggcuug

SEQ ID NO: 264 - RNA, 21 nt, Artificial Sequence
  AS sequence
aauguuagcaggauugcgggu

SEQ ID NO: 265 - RNA, 21 nt, Artificial Sequence
  AS sequence
aaacgcaguauuauuggguaa

SEQ ID NO: 266 - RNA, 21 nt, Artificial Sequence
  AS sequence
aaucgagggaauuuaaggucu

SEQ ID NO: 267 - RNA, 21 nt, Artificial Sequence
  AS sequence
aaugguguuaauuggaacgcc

SEQ ID NO: 268 - RNA, 21 nt, Artificial Sequence
  AS sequence
aagaggaaguuguagcacgau

SEQ ID NO: 269 - RNA, 21 nt, Artificial Sequence
  AS sequence
aauguauucaaggcucccuca

SEQ ID NO: 270 - RNA, 21 nt, Artificial Sequence
  AS sequence
aaaguugcaacccauaugaug

SEQ ID NO: 271 - RNA, 21 nt, Artificial Sequence
  AS sequence
aagucaucuggacugcuauug

SEQ ID NO: 272 - RNA, 21 nt, Artificial Sequence
  AS sequence
aauuuggucaucuggacugcu

SEQ ID NO: 273 - RNA, 21 nt, Artificial Sequence
  AS sequence
aaaucuuggacugagaucuuu

SEQ ID NO: 274 - RNA, 21 nt, Artificial Sequence
  AS sequence
aagcggugaaccaagacgcag

SEQ ID NO: 275 - RNA, 21 nt, Artificial Sequence
  AS sequence
aaucuucgguaguagccaauu

SEQ ID NO: 276 - RNA, 21 nt, Artificial Sequence
  AS sequence
aaauucgucugguagcucuuc

SEQ ID NO: 277 - RNA, 21 nt, Artificial Sequence
  AS sequence
aaccauaugaugccgucuuug

SEQ ID NO: 278 - RNA, 21 nt, Artificial Sequence
  AS sequence
aaaacccauaugaugccgucu

SEQ ID NO: 279 - RNA, 21 nt, Artificial Sequence
  AS sequence
aacaccaccacgaauucgucu

SEQ ID NO: 280 - RNA, 21 nt, Artificial Sequence
  AS sequence
aacgucaccaccacgaauucg

SEQ ID NO: 281 - RNA, 21 nt, Artificial Sequence
  AS sequence
aaaaauaccaucuuggacuga

SEQ ID NO: 282 - RNA, 21 nt, Artificial Sequence
  AS sequence
aauguugcgacuacgugauga

SEQ ID NO: 283 - RNA, 21 nt, Artificial Sequence
  AS sequence
aauagcaccauagggaagucc

SEQ ID NO: 284 - RNA, 21 nt, Artificial Sequence
  AS sequence
aagcaccauagggaaguccag

SEQ ID NO: 285 - RNA, 21 nt, Artificial Sequence
  AS sequence
aacauuccgaagaacgcugaa

SEQ ID NO: 286 - RNA, 21 nt, Artificial Sequence
  AS sequence
aagagagcggugaaccaagac

SEQ ID NO: 287 - RNA, 21 nt, Artificial Sequence
  AS sequence
aagcugguucaaucugucaag

SEQ ID NO: 288 - RNA, 21 nt, Artificial Sequence
  AS sequence
aaccgaagaacgcugaagcgc

SEQ ID NO: 289 - RNA, 21 nt, Artificial Sequence
  AS sequence
aacuacgugaugaggaacgag

SEQ ID NO: 290 - RNA, 21 nt, Artificial Sequence
  AS sequence
aaugccgaaagcuuguguuac

SEQ ID NO: 291 - RNA, 21 nt, Artificial Sequence
  AS sequence
aaaugcgucaauaugcuuauu

SEQ ID NO: 292 - RNA, 21 nt, Artificial Sequence
  AS sequence
aauugcggccaauguuuguaa

SEQ ID NO: 293 - RNA, 21 nt, Artificial Sequence
  AS sequence
aagugcaauuugcggccaaug

SEQ ID NO: 294 - RNA, 21 nt, Artificial Sequence
  AS sequence
aagcggccaauguuuguaauc

SEQ ID NO: 295 - RNA, 21 nt, Artificial Sequence
  AS sequence
aacuggaccacgucugccgaa

SEQ ID NO: 296 - RNA, 21 nt, Artificial Sequence
  AS sequence
aagcgcgacauuccgaagaac

SEQ ID NO: 297 - RNA, 21 nt, Artificial Sequence
  AS sequence
aaaagaacgcugaagcgcugg

SEQ ID NO: 298 - RNA, 21 nt, Artificial Sequence
  AS sequence
aagcaagagcagcaucaccgc

SEQ ID NO: 299 - RNA, 21 nt, Artificial Sequence
  AS sequence
aacgucaauaugcuuauucag

SEQ ID NO: 300 - RNA, 21 nt, Artificial Sequence
  AS sequence
aaguucccgaaggugugacuu

SEQ ID NO: 301 - RNA, 21 nt, Artificial Sequence
  AS sequence
aacguucccgaaggugugacu

SEQ ID NO: 302 - RNA, 21 nt, Artificial Sequence
  AS sequence
aauguguaggucaaccacguu

SEQ ID NO: 303 - RNA, 21 nt, Artificial Sequence
  AS sequence
aaccuguguaggucaaccacg

SEQ ID NO: 304 - RNA, 21 nt, Artificial Sequence
  AS sequence
aaugcgguaaggcuugaguuu

SEQ ID NO: 305 - RNA, 21 nt, Artificial Sequence
  AS sequence
aacacguucccgaagguguga

SEQ ID NO: 306 - RNA, 21 nt, Artificial Sequence
  AS sequence
aaggcaccuguguaggucaac

SEQ ID NO: 307 - RNA, 21 nt, Artificial Sequence
  AS sequence
aaaagucuagagcuauguaag

SEQ ID NO: 308 - RNA, 21 nt, Artificial Sequence
  AS sequence
aaaaauuaccggguuugacag

SEQ ID NO: 309 - RNA, 21 nt, Artificial Sequence
  AS sequence
aacggguuugacaguuugaaa

SEQ ID NO: 310 - RNA, 21 nt, Artificial Sequence
  AS sequence
aauaugugguaccaugucacc

SEQ ID NO: 311 - RNA, 21 nt, Artificial Sequence
  AS sequence
aagauauaugugguaccaugu

SEQ ID NO: 312 - RNA, 21 nt, Artificial Sequence
  AS sequence
aaauauaugugguaccauguc

SEQ ID NO: 313 - RNA, 21 nt, Artificial Sequence
  AS sequence
aauugcacaaugcagaaugca

SEQ ID NO: 314 - RNA, 21 nt, Artificial Sequence
  AS sequence
aaaguuugcacaaugcagaau

SEQ ID NO: 315 - RNA, 21 nt, Artificial Sequence
  AS sequence
aaauuuaguaagacguugacg

SEQ ID NO: 316 - RNA, 21 nt, Artificial Sequence
  AS sequence
aaagacguugacgugauauau

SEQ ID NO: 317 - RNA, 21 nt, Artificial Sequence
  AS sequence
aaaccgucuauucuaaacuua

SEQ ID NO: 318 - RNA, 21 nt, Artificial Sequence
  AS sequence
aauaguaagacguugacguga

SEQ ID NO: 319 - RNA, 21 nt, Artificial Sequence
  AS sequence
aaaugucaccgucuauucuaa

SEQ ID NO: 320 - RNA, 21 nt, Artificial Sequence
  AS sequence
aauucaccuaaguuggcguau

SEQ ID NO: 321 - RNA, 21 nt, Artificial Sequence
  AS sequence
aaugguaccaugucaccgucu

SEQ ID NO: 322 - RNA, 21 nt, Artificial Sequence
  AS sequence
aacacaauuaccuucaucaaa

SEQ ID NO: 323 - RNA, 21 nt, Artificial Sequence
  AS sequence
aaaccaugucaccgucuauuc

SEQ ID NO: 324 - RNA, 21 nt, Artificial Sequence
  AS sequence
aaccaugucaccgucuauucu

SEQ ID NO: 325 - RNA, 21 nt, Artificial Sequence
  AS sequence
aauguauuuaguaagacguug

SEQ ID NO: 326 - RNA, 21 nt, Artificial Sequence
  AS sequence
aacaucacaacaauuguaugu

SEQ ID NO: 327 - RNA, 21 nt, Artificial Sequence
  AS sequence
aacuaccuggcgugguuugua

SEQ ID NO: 328 - RNA, 21 nt, Artificial Sequence
  AS sequence
aaucacaacaauuguauguga

SEQ ID NO: 329 - RNA, 21 nt, Artificial Sequence
  AS sequence
aaacuaccuggcgugguuugu

SEQ ID NO: 330 - RNA, 21 nt, Artificial Sequence
  AS sequence
aacaauuguaugugacaagua

SEQ ID NO: 331 - RNA, 21 nt, Artificial Sequence
  AS sequence
aaugaaauaaucaucaucaca

SEQ ID NO: 332 - RNA, 21 nt, Artificial Sequence
  AS sequence
aaggcguacacguucaccuaa

SEQ ID NO: 333 - RNA, 21 nt, Artificial Sequence
  AS sequence
aaaugugacaaguauuucuuu

SEQ ID NO: 334 - RNA, 21 nt, Artificial Sequence
  AS sequence
aacuggucaagguuaauauag

SEQ ID NO: 335 - RNA, 21 nt, Artificial Sequence
  AS sequence
aacacaauuugggugguaugu

SEQ ID NO: 336 - RNA, 21 nt, Artificial Sequence
  AS sequence
aaaguuaacacaauuugggug

SEQ ID NO: 337 - RNA, 21 nt, Artificial Sequence
  AS sequence
aaccuggcgugguuuguauga

SEQ ID NO: 338 - RNA, 21 nt, Artificial Sequence
  AS sequence
aaaauuugggugguaugucug

SEQ ID NO: 339 - RNA, 21 nt, Artificial Sequence
  AS sequence
aaauuucgcauggcaucacag

SEQ ID NO: 340 - RNA, 21 nt, Artificial Sequence
  AS sequence
aaaacauuaaaguuugcacaa

SEQ ID NO: 341 - RNA, 21 nt, Artificial Sequence
  AS sequence
aaucacagaauuguacuguuu

SEQ ID NO: 342 - RNA, 21 nt, Artificial Sequence
  AS sequence
aagcguauacgcguaauauau

SEQ ID NO: 343 - RNA, 21 nt, Artificial Sequence
  AS sequence
aacaguuaacacaauuugggu

SEQ ID NO: 344 - RNA, 21 nt, Artificial Sequence
  AS sequence
aaacaauaccagcauuucgca

SEQ ID NO: 345 - RNA, 21 nt, Artificial Sequence
  AS sequence
aacaaaucccacuuaauguaa

SEQ ID NO: 346 - RNA, 21 nt, Artificial Sequence
  AS sequence
aaucuugauuaucuaauguca

SEQ ID NO: 347 - RNA, 21 nt, Artificial Sequence
  AS sequence
aagguuuguaugaaaucaccg

SEQ ID NO: 348 - RNA, 21 nt, Artificial Sequence
  AS sequence
aaacauuguuaguaagugcag

SEQ ID NO: 349 - RNA, 21 nt, Artificial Sequence
  AS sequence
aauuaucuaaugucaguacac

SEQ ID NO: 350 - RNA, 21 nt, Artificial Sequence
  AS sequence
aagaucuugauuaucuaaugu

SEQ ID NO: 351 - RNA, 21 nt, Artificial Sequence
  AS sequence
aaauauaggcauuaacaauga

SEQ ID NO: 352 - RNA, 21 nt, Artificial Sequence
  AS sequence
aaaguuuacauccugauuaug

SEQ ID NO: 353 - RNA, 21 nt, Artificial Sequence
  AS sequence
aaaagucagugucaacaugug

SEQ ID NO: 354 - RNA, 21 nt, Artificial Sequence
  AS sequence
aaaugugucacaauuaccuuc

SEQ ID NO: 355 - RNA, 21 nt, Artificial Sequence
  AS sequence
aagucauagaagucuuuguua

SEQ ID NO: 356 - RNA, 21 nt, Artificial Sequence
  AS sequence
aauacagccaccaucguaaca

SEQ ID NO: 357 - RNA, 21 nt, Artificial Sequence
  AS sequence
aacagcuggacaauccuuaag

SEQ ID NO: 358 - RNA, 21 nt, Artificial Sequence
  AS sequence
aaaucccacuuaauguaaggc

SEQ ID NO: 359 - RNA, 21 nt, Artificial Sequence
  AS sequence
aaacaguuaacacaauuuggg

SEQ ID NO: 360 - RNA, 21 nt, Artificial Sequence
  AS sequence
aaagcacguagugcguuuauc

SEQ ID NO: 361 - RNA, 21 nt, Artificial Sequence
  AS sequence
aagcaacauuguuaguaagug

SEQ ID NO: 362 - RNA, 21 nt, Artificial Sequence
  AS sequence
aagguuaauauaggcauuaac

SEQ ID NO: 363 - RNA, 21 nt, Artificial Sequence
  AS sequence
aagaaucuacaacaggaacuc

SEQ ID NO: 364 - RNA, 21 nt, Artificial Sequence
  AS sequence
aacauccugauuauguacaac

SEQ ID NO: 365 - RNA, 21 nt, Artificial Sequence
  AS sequence
aacuuuaucauuguagauguc

SEQ ID NO: 366 - RNA, 21 nt, Artificial Sequence
  AS sequence
aaaccagcuacuuuaucauug

SEQ ID NO: 367 - RNA, 21 nt, Artificial Sequence
  AS sequence
aaaugccuuaaagcauagacg

SEQ ID NO: 368 - RNA, 21 nt, Artificial Sequence
  AS sequence
aaaucaucaucacaacaauug

SEQ ID NO: 369 - RNA, 21 nt, Artificial Sequence
  AS sequence
aauguaaggcuuuguuaaguc

SEQ ID NO: 370 - RNA, 21 nt, Artificial Sequence
  AS sequence
aaucuucauguugguaguuag

SEQ ID NO: 371 - RNA, 21 nt, Artificial Sequence
  AS sequence
aauaaccucucuuccgugaag

SEQ ID NO: 372 - RNA, 21 nt, Artificial Sequence
  AS sequence
aaauaguagggaugacauuac

SEQ ID NO: 373 - RNA, 21 nt, Artificial Sequence
  AS sequence
aaaacuguaauaguugugucc

SEQ ID NO: 374 - RNA, 21 nt, Artificial Sequence
  AS sequence
aaugucaacauggucuugugu

SEQ ID NO: 375 - RNA, 21 nt, Artificial Sequence
  AS sequence
aaguaugucaacauggucuug

SEQ ID NO: 376 - RNA, 21 nt, Artificial Sequence
  AS sequence
aaaagguguaaauucaucuuc

SEQ ID NO: 377 - RNA, 21 nt, Artificial Sequence
  AS sequence
aaacaccugagcauugucuaa

SEQ ID NO: 378 - RNA, 21 nt, Artificial Sequence
  AS sequence
aagucuucagaggugcagauc

SEQ ID NO: 379 - RNA, 21 nt, Artificial Sequence
  AS sequence
aaacauuaccagccuguacca

SEQ ID NO: 380 - RNA, 21 nt, Artificial Sequence
  AS sequence
aaggauuggcuguaucaaccu

SEQ ID NO: 381 - RNA, 21 nt, Artificial Sequence
  AS sequence
aauacuuaggugucuuaggau

SEQ ID NO: 382 - RNA, 21 nt, Artificial Sequence
  AS sequence
aaugguugaaugcgaacaaac

SEQ ID NO: 383 - RNA, 21 nt, Artificial Sequence
  AS sequence
aacccuuaauagugaaauugg

SEQ ID NO: 384 - RNA, 21 nt, Artificial Sequence
  AS sequence
aaaccuucuaagucugugcca

SEQ ID NO: 385 - RNA, 21 nt, Artificial Sequence
  AS sequence
aaacauuaacuguaauaguug

SEQ ID NO: 386 - RNA, 21 nt, Artificial Sequence
  AS sequence
aaucauuaagaguugugguaa

SEQ ID NO: 387 - RNA, 21 nt, Artificial Sequence
  AS sequence
aauacuucauagccacaaggu

SEQ ID NO: 388 - RNA, 21 nt, Artificial Sequence
  AS sequence
aaguguuagagguucauaauu

SEQ ID NO: 389 - RNA, 21 nt, Artificial Sequence
  AS sequence
aaccaagaaauuaugauuaga

SEQ ID NO: 390 - RNA, 21 nt, Artificial Sequence
  AS sequence
aaugcgaacaaacuuauacuu

SEQ ID NO: 391 - RNA, 21 nt, Artificial Sequence
  AS sequence
aaauggugaaccauuguaaca

SEQ ID NO: 392 - RNA, 21 nt, Artificial Sequence
  AS sequence
aaucauagcacauugguaaac

SEQ ID NO: 393 - RNA, 21 nt, Artificial Sequence
  AS sequence
aaaagagacacagucauaauc

SEQ ID NO: 394 - RNA, 21 nt, Artificial Sequence
  AS sequence
aagucauuaagaguuguggua

SEQ ID NO: 395 - RNA, 21 nt, Artificial Sequence
  AS sequence
aacaagguuaaagucauuaag

SEQ ID NO: 396 - RNA, 21 nt, Artificial Sequence
  AS sequence
aaguucauaauuguacuucau

SEQ ID NO: 397 - RNA, 21 nt, Artificial Sequence
  AS sequence
aagagguucauaauuguacuu

SEQ ID NO: 398 - RNA, 21 nt, Artificial Sequence
  AS sequence
aaacuacccaauaugguacgu

SEQ ID NO: 399 - RNA, 21 nt, Artificial Sequence
  AS sequence
aauucagaggugcagaucaca

SEQ ID NO: 400 - RNA, 21 nt, Artificial Sequence
  AS sequence
aauaaacaccagauggugaac

SEQ ID NO: 401 - RNA, 21 nt, Artificial Sequence
  AS sequence
aauguaauaagcaucuuguag

SEQ ID NO: 402 - RNA, 21 nt, Artificial Sequence
  AS sequence
aacucaccuacugucuuauua

SEQ ID NO: 403 - RNA, 21 nt, Artificial Sequence
  AS sequence
aaacccuuaaggguugucugc

SEQ ID NO: 404 - RNA, 21 nt, Artificial Sequence
  AS sequence
aacaauacaaaguuucuuuag

SEQ ID NO: 405 - RNA, 21 nt, Artificial Sequence
  AS sequence
aauaauaggaccuuuguauuc

SEQ ID NO: 406 - RNA, 21 nt, Artificial Sequence
  AS sequence
aaaacuuguggguauuuccac

SEQ ID NO: 407 - RNA, 21 nt, Artificial Sequence
  AS sequence
aauuguuaucugcccauuuaa

SEQ ID NO: 408 - RNA, 21 nt, Artificial Sequence
  AS sequence
aauauuuguuggaguguuaac

SEQ ID NO: 409 - RNA, 21 nt, Artificial Sequence
  AS sequence
aacuauuuguuggaguguuaa

SEQ ID NO: 410 - RNA, 21 nt, Artificial Sequence
  AS sequence
aaaaguuagcagcuucaccag

SEQ ID NO: 411 - RNA, 21 nt, Artificial Sequence
  AS sequence
aaggguugucugcuguugucc

SEQ ID NO: 412 - RNA, 21 nt, Artificial Sequence
  AS sequence
aacccuuaaggguugucugcu

SEQ ID NO: 413 - RNA, 21 nt, Artificial Sequence
  AS sequence
aauaauuaccaguguacucac

SEQ ID NO: 414 - RNA, 21 nt, Artificial Sequence
  AS sequence
aaauguuuauagugaccacac

SEQ ID NO: 415 - RNA, 21 nt, Artificial Sequence
  AS sequence
aaacuuuguaaguaaagcacc

SEQ ID NO: 416 - RNA, 21 nt, Artificial Sequence
  AS sequence
aaguauucugaggacuuugua

SEQ ID NO: 417 - RNA, 21 nt, Artificial Sequence
  AS sequence
aacaagauaacaguuguuauc

SEQ ID NO: 418 - RNA, 21 nt, Artificial Sequence
  AS sequence
aaaacuucaacucuauuuguu

SEQ ID NO: 419 - RNA, 21 nt, Artificial Sequence
  AS sequence
aaucugaacaccuuucuuaaa

SEQ ID NO: 420 - RNA, 21 nt, Artificial Sequence
  AS sequence
aacaugcuuaaguucauacug

SEQ ID NO: 421 - RNA, 15 nt, Artificial Sequence
  SS sequence
gaucacauuggcacc

SEQ ID NO: 422 - RNA, 15 nt, Artificial Sequence
  SS sequence
cacaagcuuucggca

SEQ ID NO: 423 - RNA, 15 nt, Artificial Sequence
  SS sequence
cagcaaacugugacu

SEQ ID NO: 424 - RNA, 15 nt, Artificial Sequence
  SS sequence
guuuacccaauaaua

SEQ ID NO: 425 - RNA, 15 nt, Artificial Sequence
  SS sequence
cgcucucacucaaca

SEQ ID NO: 426 - RNA, 15 nt, Artificial Sequence
  SS sequence
acauggcaaggaaga

SEQ ID NO: 427 - RNA, 15 nt, Artificial Sequence
  SS sequence
ggaagaccuuaaauu

SEQ ID NO: 428 - RNA, 15 nt, Artificial Sequence
  SS sequence
gcguuccaauuaaca

SEQ ID NO: 429 - RNA, 15 nt, Artificial Sequence
  SS sequence
uacuaccuaggaacu

SEQ ID NO: 430 - RNA, 15 nt, Artificial Sequence
  SS sequence
auggugcuaacaaag

SEQ ID NO: 431 - RNA, 15 nt, Artificial Sequence
  SS sequence
agggagccuugaaua

SEQ ID NO: 432 - RNA, 15 nt, Artificial Sequence
  SS sequence
uuggcacccgcaauc

SEQ ID NO: 433 - RNA, 15 nt, Artificial Sequence
  SS sequence
gcaauccugcuaaca

SEQ ID NO: 434 - RNA, 15 nt, Artificial Sequence
  SS sequence
auccugcuaacaaug

SEQ ID NO: 435 - RNA, 15 nt, Artificial Sequence
  SS sequence
cuuccucaaggaaca

SEQ ID NO: 436 - RNA, 15 nt, Artificial Sequence
  SS sequence
cuucuacgcagaagg

SEQ ID NO: 437 - RNA, 15 nt, Artificial Sequence
  SS sequence
aggcggcagucaagc

SEQ ID NO: 438 - RNA, 15 nt, Artificial Sequence
  SS sequence
acaguucaagaaauu

SEQ ID NO: 439 - RNA, 15 nt, Artificial Sequence
  SS sequence
gaauggcuggcaaug

SEQ ID NO: 440 - RNA, 15 nt, Artificial Sequence
  SS sequence
uaaaggccaacaaca

SEQ ID NO: 441 - RNA, 15 nt, Artificial Sequence
  SS sequence
cugucacuaagaaau

SEQ ID NO: 442 - RNA, 15 nt, Artificial Sequence
  SS sequence
cugaggcuucuaaga

SEQ ID NO: 443 - RNA, 15 nt, Artificial Sequence
  SS sequence
aggcuucuaagaagc

SEQ ID NO: 444 - RNA, 15 nt, Artificial Sequence
  SS sequence
guacugccacuaaag

SEQ ID NO: 445 - RNA, 15 nt, Artificial Sequence
  SS sequence
uacugccacuaaagc

SEQ ID NO: 446 - RNA, 15 nt, Artificial Sequence
  SS sequence
cuaaagcauacaaug

SEQ ID NO: 447 - RNA, 15 nt, Artificial Sequence
  SS sequence
caaacccaaggaaau

SEQ ID NO: 448 - RNA, 15 nt, Artificial Sequence
  SS sequence
acuaaucagacaagg

SEQ ID NO: 449 - RNA, 15 nt, Artificial Sequence
  SS sequence
aucagacaaggaacu

SEQ ID NO: 450 - RNA, 15 nt, Artificial Sequence
  SS sequence
cauuggccgcaaauu

SEQ ID NO: 451 - RNA, 15 nt, Artificial Sequence
  SS sequence
cauuggcauggaagu

SEQ ID NO: 452 - RNA, 15 nt, Artificial Sequence
  SS sequence
caggugccaucaaau

SEQ ID NO: 453 - RNA, 15 nt, Artificial Sequence
  SS sequence
aggugccaucaaauu

SEQ ID NO: 454 - RNA, 15 nt, Artificial Sequence
  SS sequence
aagaaacagcaaacu

SEQ ID NO: 455 - RNA, 15 nt, Artificial Sequence
  SS sequence
ugauuucuccaaaca

SEQ ID NO: 456 - RNA, 15 nt, Artificial Sequence
  SS sequence
uuucuccaaacaauu

SEQ ID NO: 457 - RNA, 15 nt, Artificial Sequence
  SS sequence
cauuacguuuggugg

SEQ ID NO: 458 - RNA, 15 nt, Artificial Sequence
  SS sequence
aaccagaauggagaa

SEQ ID NO: 459 - RNA, 15 nt, Artificial Sequence
  SS sequence
aauaauacugcgucu

SEQ ID NO: 460 - RNA, 15 nt, Artificial Sequence
  SS sequence
gaagaccuuaaauuc

SEQ ID NO: 461 - RNA, 15 nt, Artificial Sequence
  SS sequence
gaccaaauuggcuac

SEQ ID NO: 462 - RNA, 15 nt, Artificial Sequence
  SS sequence
caagaugguauuucu

SEQ ID NO: 463 - RNA, 15 nt, Artificial Sequence
  SS sequence
aacaaugcugcaauc

SEQ ID NO: 464 - RNA, 15 nt, Artificial Sequence
  SS sequence
gaacaacauugccaa

SEQ ID NO: 465 - RNA, 15 nt, Artificial Sequence
  SS sequence
caagaaauucaacuc

SEQ ID NO: 466 - RNA, 15 nt, Artificial Sequence
  SS sequence
aaccagcuugagagc

SEQ ID NO: 467 - RNA, 15 nt, Artificial Sequence
  SS sequence
aagaaaucugcugcu

SEQ ID NO: 468 - RNA, 15 nt, Artificial Sequence
  SS sequence
aagaagccucggcaa

SEQ ID NO: 469 - RNA, 15 nt, Artificial Sequence
  SS sequence
aucauauggguugca

SEQ ID NO: 470 - RNA, 15 nt, Artificial Sequence
  SS sequence
ucauauggguugcaa

SEQ ID NO: 471 - RNA, 15 nt, Artificial Sequence
  SS sequence
cauauggguugcaac

SEQ ID NO: 472 - RNA, 15 nt, Artificial Sequence
  SS sequence
ccucuucucguuccu

SEQ ID NO: 473 - RNA, 15 nt, Artificial Sequence
  SS sequence
gccucuucucguucc

SEQ ID NO: 474 - RNA, 15 nt, Artificial Sequence
  SS sequence
cgcaauccugcuaac

SEQ ID NO: 475 - RNA, 15 nt, Artificial Sequence
  SS sequence
cccaauaauacugcg

SEQ ID NO: 476 - RNA, 15 nt, Artificial Sequence
  SS sequence
ccuuaaauucccucg

SEQ ID NO: 477 - RNA, 15 nt, Artificial Sequence
  SS sequence
guuccaauuaacacc

SEQ ID NO: 478 - RNA, 15 nt, Artificial Sequence
  SS sequence
gugcuacaacuuccu

SEQ ID NO: 479 - RNA, 15 nt, Artificial Sequence
  SS sequence
gggagccuugaauac

SEQ ID NO: 480 - RNA, 15 nt, Artificial Sequence
  SS sequence
cauauggguugcaac

SEQ ID NO: 481 - RNA, 15 nt, Artificial Sequence
  SS sequence
uagcaguccagauga

SEQ ID NO: 482 - RNA, 15 nt, Artificial Sequence
  SS sequence
aguccagaugaccaa

SEQ ID NO: 483 - RNA, 15 nt, Artificial Sequence
  SS sequence
gaucucaguccaaga

SEQ ID NO: 484 - RNA, 15 nt, Artificial Sequence
  SS sequence
cgucuugguucaccg

SEQ ID NO: 485 - RNA, 15 nt, Artificial Sequence
  SS sequence
uggcuacuaccgaag

SEQ ID NO: 486 - RNA, 15 nt, Artificial Sequence
  SS sequence
gagcuaccagacgaa

SEQ ID NO: 487 - RNA, 15 nt, Artificial Sequence
  SS sequence
agacggcaucauaug

SEQ ID NO: 488 - RNA, 15 nt, Artificial Sequence
  SS sequence
cggcaucauaugggu

SEQ ID NO: 489 - RNA, 15 nt, Artificial Sequence
  SS sequence
cgaauucgugguggu

SEQ ID NO: 490 - RNA, 15 nt, Artificial Sequence
  SS sequence
auucgugguggugac

SEQ ID NO: 491 - RNA, 15 nt, Artificial Sequence
  SS sequence
guccaagaugguauu

SEQ ID NO: 492 - RNA, 15 nt, Artificial Sequence
  SS sequence
ucacguagucgcaac

SEQ ID NO: 493 - RNA, 15 nt, Artificial Sequence
  SS sequence
cuucccuauggugcu

SEQ ID NO: 494 - RNA, 15 nt, Artificial Sequence
  SS sequence
gacuucccuauggug

SEQ ID NO: 495 - RNA, 15 nt, Artificial Sequence
  SS sequence
agcguucuucggaau

SEQ ID NO: 496 - RNA, 15 nt, Artificial Sequence
  SS sequence
uugguucaccgcucu

SEQ ID NO: 497 - RNA, 15 nt, Artificial Sequence
  SS sequence
gacagauugaaccag

SEQ ID NO: 498 - RNA, 15 nt, Artificial Sequence
  SS sequence
cuucagcguucuucg

SEQ ID NO: 499 - RNA, 15 nt, Artificial Sequence
  SS sequence
guuccucaucacgua

SEQ ID NO: 500 - RNA, 15 nt, Artificial Sequence
  SS sequence
acacaagcuuucggc

SEQ ID NO: 501 - RNA, 15 nt, Artificial Sequence
  SS sequence
aagcauauugacgca

SEQ ID NO: 502 - RNA, 15 nt, Artificial Sequence
  SS sequence
caaacauuggccgca

SEQ ID NO: 503 - RNA, 15 nt, Artificial Sequence
  SS sequence
uggccgcaaauugca

SEQ ID NO: 504 - RNA, 15 nt, Artificial Sequence
  SS sequence
uacaaacauuggccg

SEQ ID NO: 505 - RNA, 15 nt, Artificial Sequence
  SS sequence
ggcagacguggucca

SEQ ID NO: 506 - RNA, 15 nt, Artificial Sequence
  SS sequence
cuucggaaugucgcg

SEQ ID NO: 507 - RNA, 15 nt, Artificial Sequence
  SS sequence
gcgcuucagcguucu

SEQ ID NO: 508 - RNA, 15 nt, Artificial Sequence
  SS sequence
gugaugcugcucuug

SEQ ID NO: 509 - RNA, 15 nt, Artificial Sequence
  SS sequence
aauaagcauauugac

SEQ ID NO: 510 - RNA, 15 nt, Artificial Sequence
  SS sequence
ucacaccuucgggaa

SEQ ID NO: 511 - RNA, 15 nt, Artificial Sequence
  SS sequence
cacaccuucgggaac

SEQ ID NO: 512 - RNA, 15 nt, Artificial Sequence
  SS sequence
gugguugaccuacac

SEQ ID NO: 513 - RNA, 15 nt, Artificial Sequence
  SS sequence
gguugaccuacacag

SEQ ID NO: 514 - RNA, 15 nt, Artificial Sequence
  SS sequence
cucaagccuuaccgc

SEQ ID NO: 515 - RNA, 15 nt, Artificial Sequence
  SS sequence
caccuucgggaacgu

SEQ ID NO: 516 - RNA, 15 nt, Artificial Sequence
  SS sequence
gaccuacacaggugc

SEQ ID NO: 517 - RNA, 15 nt, Artificial Sequence
  SS sequence
acauagcucuagacu

SEQ ID NO: 518 - RNA, 15 nt, Artificial Sequence
  SS sequence
ucaaacccgguaauu

SEQ ID NO: 519 - RNA, 15 nt, Artificial Sequence
  SS sequence
caaacugucaaaccc

SEQ ID NO: 520 - RNA, 15 nt, Artificial Sequence
  SS sequence
gacaugguaccacau

SEQ ID NO: 521 - RNA, 15 nt, Artificial Sequence
  SS sequence
ugguaccacauauau

SEQ ID NO: 522 - RNA, 15 nt, Artificial Sequence
  SS sequence
augguaccacauaua

SEQ ID NO: 523 - RNA, 15 nt, Artificial Sequence
  SS sequence
auucugcauugugca

SEQ ID NO: 524 - RNA, 15 nt, Artificial Sequence
  SS sequence
cugcauugugcaaac

SEQ ID NO: 525 - RNA, 15 nt, Artificial Sequence
  SS sequence
caacgucuuacuaaa

SEQ ID NO: 526 - RNA, 15 nt, Artificial Sequence
  SS sequence
uaucacgucaacguc

SEQ ID NO: 527 - RNA, 15 nt, Artificial Sequence
  SS sequence
guuuagaauagacgg

SEQ ID NO: 528 - RNA, 15 nt, Artificial Sequence
  SS sequence
cgucaacgucuuacu

SEQ ID NO: 529 - RNA, 15 nt, Artificial Sequence
  SS sequence
gaauagacggugaca

SEQ ID NO: 530 - RNA, 15 nt, Artificial Sequence
  SS sequence
cgccaacuuagguga

SEQ ID NO: 531 - RNA, 15 nt, Artificial Sequence
  SS sequence
cggugacaugguacc

SEQ ID NO: 532 - RNA, 15 nt, Artificial Sequence
  SS sequence
gaugaagguaauugu

SEQ ID NO: 533 - RNA, 15 nt, Artificial Sequence
  SS sequence
uagacggugacaugg

SEQ ID NO: 534 - RNA, 15 nt, Artificial Sequence
  SS sequence
auagacggugacaug

SEQ ID NO: 535 - RNA, 15 nt, Artificial Sequence
  SS sequence
cgucuuacuaaauac

SEQ ID NO: 536 - RNA, 15 nt, Artificial Sequence
  SS sequence
uacaauuguugugau

SEQ ID NO: 537 - RNA, 15 nt, Artificial Sequence
  SS sequence
aaaccacgccaggua

SEQ ID NO: 538 - RNA, 15 nt, Artificial Sequence
  SS sequence
cauacaauuguugug

SEQ ID NO: 539 - RNA, 15 nt, Artificial Sequence
  SS sequence
aaccacgccagguag

SEQ ID NO: 540 - RNA, 15 nt, Artificial Sequence
  SS sequence
uugucacauacaauu

SEQ ID NO: 541 - RNA, 15 nt, Artificial Sequence
  SS sequence
gaugaugauuauuuc

SEQ ID NO: 542 - RNA, 15 nt, Artificial Sequence
  SS sequence
ggugaacguguacgc

SEQ ID NO: 543 - RNA, 15 nt, Artificial Sequence
  SS sequence
gaaauacuugucaca

SEQ ID NO: 544 - RNA, 15 nt, Artificial Sequence
  SS sequence
uauuaaccuugacca

SEQ ID NO: 545 - RNA, 15 nt, Artificial Sequence
  SS sequence
uaccacccaaauugu

SEQ ID NO: 546 - RNA, 15 nt, Artificial Sequence
  SS sequence
ccaaauuguguuaac

SEQ ID NO: 547 - RNA, 15 nt, Artificial Sequence
  SS sequence
uacaaaccacgccag

SEQ ID NO: 548 - RNA, 15 nt, Artificial Sequence
  SS sequence
acauaccacccaaau

SEQ ID NO: 549 - RNA, 15 nt, Artificial Sequence
  SS sequence
ugaugccaugcgaaa

SEQ ID NO: 550 - RNA, 15 nt, Artificial Sequence
  SS sequence
ugcaaacuuuaaugu

SEQ ID NO: 551 - RNA, 15 nt, Artificial Sequence
  SS sequence
caguacaauucugug

SEQ ID NO: 552 - RNA, 15 nt, Artificial Sequence
  SS sequence
uauuacgcguauacg

SEQ ID NO: 553 - RNA, 15 nt, Artificial Sequence
  SS sequence
caaauuguguuaacu

SEQ ID NO: 554 - RNA, 15 nt, Artificial Sequence
  SS sequence
gaaaugcugguauug

SEQ ID NO: 555 - RNA, 15 nt, Artificial Sequence
  SS sequence
cauuaagugggauuu

SEQ ID NO: 556 - RNA, 15 nt, Artificial Sequence
  SS sequence
cauuagauaaucaag

SEQ ID NO: 557 - RNA, 15 nt, Artificial Sequence
  SS sequence
ugauuucauacaaac

SEQ ID NO: 558 - RNA, 15 nt, Artificial Sequence
  SS sequence
cacuuacuaacaaug

SEQ ID NO: 559 - RNA, 15 nt, Artificial Sequence
  SS sequence
uacugacauuagaua

SEQ ID NO: 560 - RNA, 15 nt, Artificial Sequence
  SS sequence
uuagauaaucaagau

SEQ ID NO: 561 - RNA, 15 nt, Artificial Sequence
  SS sequence
uuguuaaugccuaua

SEQ ID NO: 562 - RNA, 15 nt, Artificial Sequence
  SS sequence
aaucaggauguaaac

SEQ ID NO: 563 - RNA, 15 nt, Artificial Sequence
  SS sequence
auguugacacugacu

SEQ ID NO: 564 - RNA, 15 nt, Artificial Sequence
  SS sequence
gguaauugugacaca

SEQ ID NO: 565 - RNA, 15 nt, Artificial Sequence
  SS sequence
caaagacuucuauga

SEQ ID NO: 566 - RNA, 15 nt, Artificial Sequence
  SS sequence
uacgaugguggcugu

SEQ ID NO: 567 - RNA, 15 nt, Artificial Sequence
  SS sequence
aaggauuguccagcu

SEQ ID NO: 568 - RNA, 15 nt, Artificial Sequence
  SS sequence
uuacauuaaguggga

SEQ ID NO: 569 - RNA, 15 nt, Artificial Sequence
  SS sequence
aaauuguguuaacug

SEQ ID NO: 570 - RNA, 15 nt, Artificial Sequence
  SS sequence
aaacgcacuacgugc

SEQ ID NO: 571 - RNA, 15 nt, Artificial Sequence
  SS sequence
uuacuaacaauguug

SEQ ID NO: 572 - RNA, 15 nt, Artificial Sequence
  SS sequence
aaugccuauauuaac

SEQ ID NO: 573 - RNA, 15 nt, Artificial Sequence
  SS sequence
uuccuguuguagauu

SEQ ID NO: 574 - RNA, 15 nt, Artificial Sequence
  SS sequence
guacauaaucaggau

SEQ ID NO: 575 - RNA, 15 nt, Artificial Sequence
  SS sequence
aucuacaaugauaaa

SEQ ID NO: 576 - RNA, 15 nt, Artificial Sequence
  SS sequence
ugauaaaguagcugg

SEQ ID NO: 577 - RNA, 15 nt, Artificial Sequence
  SS sequence
cuaugcuuuaaggca

SEQ ID NO: 578 - RNA, 15 nt, Artificial Sequence
  SS sequence
uuguugugaugauga

SEQ ID NO: 579 - RNA, 15 nt, Artificial Sequence
  SS sequence
uuaacaaagccuuac

SEQ ID NO: 580 - RNA, 15 nt, Artificial Sequence
  SS sequence
acuaccaacaugaag

SEQ ID NO: 581 - RNA, 15 nt, Artificial Sequence
  SS sequence
cacggaagagagguu

SEQ ID NO: 582 - RNA, 15 nt, Artificial Sequence
  SS sequence
augucaucccuacua

SEQ ID NO: 583 - RNA, 17 nt, Artificial Sequence
  SS sequence
acacaacuauuacaguu

SEQ ID NO: 584 - RNA, 17 nt, Artificial Sequence
  SS sequence
acaagaccauguugaca

SEQ ID NO: 585 - RNA, 17 nt, Artificial Sequence
  SS sequence
agaccauguugacauac

SEQ ID NO: 586 - RNA, 17 nt, Artificial Sequence
  SS sequence
agaugaauuuacaccuu

SEQ ID NO: 587 - RNA, 17 nt, Artificial Sequence
  SS sequence
agacaaugcucaggugu

SEQ ID NO: 588 - RNA, 17 nt, Artificial Sequence
  SS sequence
ucugcaccucugaagac

SEQ ID NO: 589 - RNA, 17 nt, Artificial Sequence
  SS sequence
guacaggcugguaaugu

SEQ ID NO: 590 - RNA, 17 nt, Artificial Sequence
  SS sequence
guugauacagccaaucc

SEQ ID NO: 591 - RNA, 17 nt, Artificial Sequence
  SS sequence
ccuaagacaccuaagua

SEQ ID NO: 592 - RNA, 17 nt, Artificial Sequence
  SS sequence
uuguucgcauucaacca

SEQ ID NO: 593 - RNA, 17 nt, Artificial Sequence
  SS sequence
aauuucacuauuaaggg

SEQ ID NO: 594 - RNA, 17 nt, Artificial Sequence
  SS sequence
gcacagacuuagaaggu

SEQ ID NO: 595 - RNA, 17 nt, Artificial Sequence
  SS sequence
acuauuacaguuaaugu

SEQ ID NO: 596 - RNA, 17 nt, Artificial Sequence
  SS sequence
accacaacucuuaauga

SEQ ID NO: 597 - RNA, 17 nt, Artificial Sequence
  SS sequence
cuuguggcuaugaagua

SEQ ID NO: 598 - RNA, 17 nt, Artificial Sequence
  SS sequence
uuaugaaccucuaacac

SEQ ID NO: 599 - RNA, 17 nt, Artificial Sequence
  SS sequence
uaaucauaauuucuugg

SEQ ID NO: 600 - RNA, 17 nt, Artificial Sequence
  SS sequence
guauaaguuuguucgca

SEQ ID NO: 601 - RNA, 17 nt, Artificial Sequence
  SS sequence
uuacaaugguucaccau

SEQ ID NO: 602 - RNA, 17 nt, Artificial Sequence
  SS sequence
uuaccaaugugcuauga

SEQ ID NO: 603 - RNA, 17 nt, Artificial Sequence
  SS sequence
uuaugacugugucucuu

SEQ ID NO: 604 - RNA, 17 nt, Artificial Sequence
  SS sequence
ccacaacucuuaaugac

SEQ ID NO: 605 - RNA, 17 nt, Artificial Sequence
  SS sequence
uaaugacuuuaaccuug

SEQ ID NO: 606 - RNA, 17 nt, Artificial Sequence
  SS sequence
gaaguacaauuaugaac

SEQ ID NO: 607 - RNA, 17 nt, Artificial Sequence
  SS sequence
guacaauuaugaaccuc

SEQ ID NO: 608 - RNA, 17 nt, Artificial Sequence
  SS sequence
guaccauauuggguagu

SEQ ID NO: 609 - RNA, 17 nt, Artificial Sequence
  SS sequence
ugaucugcaccucugaa

SEQ ID NO: 610 - RNA, 17 nt, Artificial Sequence
  SS sequence
ucaccaucugguguuua

SEQ ID NO: 611 - RNA, 17 nt, Artificial Sequence
  SS sequence
acaagaugcuuauuaca

SEQ ID NO: 612 - RNA, 17 nt, Artificial Sequence
  SS sequence
auaagacaguaggugag

SEQ ID NO: 613 - RNA, 17 nt, Artificial Sequence
  SS sequence
agacaacccuuaagggu

SEQ ID NO: 614 - RNA, 17 nt, Artificial Sequence
  SS sequence
aaagaaacuuuguauug

SEQ ID NO: 615 - RNA, 17 nt, Artificial Sequence
  SS sequence
auacaaagguccuauua

SEQ ID NO: 616 - RNA, 17 nt, Artificial Sequence
  SS sequence
ggaaauacccacaaguu

SEQ ID NO: 617 - RNA, 17 nt, Artificial Sequence
  SS sequence
aaaugggcagauaacaa

SEQ ID NO: 618 - RNA, 17 nt, Artificial Sequence
  SS sequence
uaacacuccaacaaaua

SEQ ID NO: 619 - RNA, 17 nt, Artificial Sequence
  SS sequence
aacacuccaacaaauag

SEQ ID NO: 620 - RNA, 17 nt, Artificial Sequence
  SS sequence
ggugaagcugcuaacuu

SEQ ID NO: 621 - RNA, 17 nt, Artificial Sequence
  SS sequence
acaacagcagacaaccc

SEQ ID NO: 622 - RNA, 17 nt, Artificial Sequence
  SS sequence
cagacaacccuuaaggg

SEQ ID NO: 623 - RNA, 17 nt, Artificial Sequence
  SS sequence
gaguacacugguaauua

SEQ ID NO: 624 - RNA, 17 nt, Artificial Sequence
  SS sequence
guggucacuauaaacau

SEQ ID NO: 625 - RNA, 17 nt, Artificial Sequence
  SS sequence
ugcuuuacuuacaaagu

SEQ ID NO: 626 - RNA, 17 nt, Artificial Sequence
  SS sequence
caaaguccucagaauac

SEQ ID NO: 627 - RNA, 17 nt, Artificial Sequence
  SS sequence
uaacaacuguuaucuug

SEQ ID NO: 628 - RNA, 17 nt, Artificial Sequence
  SS sequence
caaauagaguugaaguu

SEQ ID NO: 629 - RNA, 17 nt, Artificial Sequence
  SS sequence
uaagaaagguguucaga

SEQ ID NO: 630 - RNA, 17 nt, Artificial Sequence
  SS sequence
guaugaacuuaagcaug